SEQUENCE LISTING (23 sequences)

SEQ ID NO: 1 - DNA, 939 bp, Homo sapiens
  CDS (1)..(939)
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctaa

SEQ ID NO: 2 - protein, 312 aa, Homo sapiens
MAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALEENELTSLAEDLLGLTDPNGGLA

SEQ ID NO: 3 - DNA, 31 bp, Artificial Sequence
  Synthetic oligonucleotide
ggatccactagtatggcagctgggcaaaatg

SEQ ID NO: 4 - DNA, 29 bp, Artificial Sequence
  Synthetic oligonucleotide
gacgcgtgctagcggccaggccgccattg

SEQ ID NO: 5 - DNA, 30 bp, Artificial Sequence
  Synthetic oligonucleotide
tgcatcaagcttatggtgagcaagggcgag

SEQ ID NO: 6 - DNA, 32 bp, Artificial Sequence
  Synthetic oligonucleotide
ggatccactagtcttgtacagctcgtccatgc

SEQ ID NO: 7 - DNA, 35 bp, Artificial Sequence
  Synthetic oligonucleotide
cagctgacgcgtccaccatggtgagcaagggcgag

SEQ ID NO: 8 - DNA, 29 bp, Artificial Sequence
  Synthetic oligonucleotide
gcgcgtcgacttaggccaggccgccattg

SEQ ID NO: 9 - DNA, 34 bp, Artificial Sequence
  Synthetic oligonucleotide
gacgcgtgctagcatggtgagcaagggcgaggag

SEQ ID NO: 10 - DNA, 31 bp, Artificial Sequence
  Synthetic oligonucleotide
actgcagatatccttgtacagctcgtccatg

SEQ ID NO: 11 - DNA, 35 bp, Artificial Sequence
  Synthetic oligonucleotide
cagctgacgcgtccaccatggcagctgggcaaaat

SEQ ID NO: 12 - DNA, 32 bp, Artificial Sequence
  Synthetic oligonucleotide
gcgcgtcgacttacttgtacagctcgtccatg

SEQ ID NO: 13 - DNA, 717 bp, Artificial Sequence
  Synthetic EYFP DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaag

SEQ ID NO: 14 - protein, 239 aa, Artificial Sequence
  Synthetic Construct
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 15 - DNA, 1662 bp, Artificial Sequence
  Synthetic EYFP-TRADD construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagactagtatggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggcctaa

SEQ ID NO: 16 - protein, 553 aa, Artificial Sequence [sequence omitted]

SEQ ID NO: 17 - DNA, 1662 bp, Artificial Sequence
  Synthetic TRADD-EYFP construct
atggcagctgggcaaaatgggcacgaagagtgggtgggcagcgcatacctgtttgtggagtcctcgctggacaaggtggtcctgtcggatgcctacgcgcacccccagcagaaggtggcagtgtacagggctctgcaggctgccttggcagagagcggcgggagcccggacgtgctgcagatgctgaagatccaccgcagcgacccgcagctgatcgtgcagctgcgattctgcgggcggcagccctgtggccgcttcctccgcgcctaccgcgagggggcgctgcgcgccgcgctgcagaggagcctggcggccgcgctcgcccagcactcggtgccgctgcaactggagctgcgcgccggcgccgagcggctggacgctttgctggcggacgaggagcgctgtttgagttgcatcctagcccagcagcccgaccggctccgggatgaagaactggctgagctggaggatgcgctgcgaaatctgaagtgcggctcgggggcccggggtggcgacggggaggtcgcttcggcccccttgcagcccccggtgccctctctgtcggaggtgaagccgccgccgccgccgccacctgcccagacttttctgttccagggtcagcctgtagtgaatcggccgctgagcctgaaggaccaacagacgttcgcgcgctctgtgggtctcaaatggcgcaaggtggggcgctcactgcagcgaggctgccgggcgctgcgggacccggcgctggactcgctggcctacgagtacgagcgcgagggactgtacgagcaggccttccagctgctgcggcgcttcgtgcaggccgagggccgccgcgccacgctgcagcgcctggtggaggcactcgaggagaacgagctcaccagcctggcagaggacttgctgggcctgaccgatcccaatggcggcctggccgctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 18 - protein, 553 aa, Artificial Sequence [sequence omitted]

SEQ ID NO: 19 - DNA, 1266 bp, Homo sapiens
  CDS (1)..(1266)
atgaacttcgggttcagcttgattttcctggtcctggtgctgaagggcgtgcagtgcgaggtgaagctggtgccacgcggatcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcttggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgaggcccgtga

SEQ ID NO: 20 - protein, 421 aa, Homo sapiens
MNFGFSLIFLVLVLKGVQCEVKLVPRGSQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLLGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 21 - DNA, 53 bp, Artificial Sequence
  Synthetic oligonucleotide
ggggacaagtttgtacaaaaaagcaggctgccaccatgaacttcgggttcagc

SEQ ID NO: 22 - DNA, 47 bp, Artificial Sequence
  Synthetic oligonucleotide
ggggaccactttgtacaagaaagctgggttcacgggcctcgctgcag

SEQ ID NO: 23 - DNA, 6429 bp, Artificial Sequence
  Synthetic pTRE2-DR3 expression vector
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggccgcatcgataagcttgtcgacgatatcacaagtttgtacaaaaaagcaggctgccaccatgaacttcgggttcagcttgattttcctggtcctggtgctgaagggcgtgcagtgcgaggtgaagctggtgccacgcggatcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcttggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgaggcccgtgaacccagctttcttgtacaaagtggtgatatctctagagctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcactcgagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgc

(19) United States
(12) Patent Application Publication
(10) Pub. No.: US 2011/0319287 A1
(43) Pub. Date: Dec. 29, 2011

(54) MODULATOR ASSAY

(75) Inventors: Aurelie Florence Chedal-bornu goiller, Plan-les-Ouates (CH); Vincent Mutel, Plan-les-Ouates (CH); Robert Johannes Luetjens, Plan-les-Ouates (CH)

(73) Assignee: ADDEX PHARMA S.A., Plan-les-Ouates (CH)

(21) Appl. No.: 13/145,248

(22) PCT Filed: Jan. 19, 2010
(86) PCT No.: PCT/IB10/00142

§ 371 Date: Sep. 6, 2011

(30) Foreign Application Priority Data
Jan. 19, 2009 (GB) GB 0900860.8

(51) Int. Cl. C40B 30/04; C07D 209/46; C07D 487/04; G01N 33/566; C07K 16/24

(52) U.S. Cl. 506/9; 435/7.21; 530/387.3; 544/346; 548/512

(57) ABSTRACT

The present invention relates inter alia to methods for identifying modulators of tumour necrosis factor (TNF) signalling. In one aspect, the method involves identifying an agent that modulates a signalling pathway mediated by TNF, comprising the steps of providing a host cell comprising TNF receptor 1 (TNFR1)-associated death domain (TRADD) linked to a reporter molecule, and determining at least one cellular characteristic detectable by the reporter molecule in the host cell in the presence of TNF and in the presence and absence of a candidate modulator. The invention is suitable for high-throughput screening (HTS) and high-content screening (HCS), or a combination of HTS and HCS.

The present invention relates inter alia to methods for detecting modulators of tumour necrosis factor (TNF) signalling, including modulators of TNF-like molecule signalling and/or TNF homologue signalling.

TNF is a pleiotropic cytokine involved in a broad range of biological activities, including inflammation, cell differentiation, survival, and cell death. It is a polypeptide hormone released from macrophages and other cells via activation of mitogen-activated protein (MAP) kinases. TNF also stimulates the synthesis of acute-phase proteins. TNF-induced cellular responses are mediated by TNF engaging with TNF receptors (TNFRs) such as TNF receptor 1 (TNFR1) and TNF receptor 2 (TNFR2). Most TNFRs are type 1 membrane proteins although some are anchored to the plasma membrane or are secreted as soluble proteins (see Wilson et al., 2009, Nature Immunol. 10: 348-355, for review).

The molecular mechanisms that regulate TNF-mediated responses are complex and varied. Based on cell culture work and studies with receptor knockout mice, it is known that both the pro-inflammatory and the programmed cell death pathways that are activated by TNF and associated with tissue injury, are largely mediated through TNFR1. The consequences of TNFR2 signalling are less well characterised, but TNFR2 has been shown to mediate signals that promote tissue repair and angiogenesis.

Several TNFRs have a conserved 80 amino acid death domain (DD) motif in their cytoplasmic tail and are referred to as death receptors (DRs). Upon binding to their ligands, the DRs recruit one of two important DD-containing adaptor (or accessory) proteins TNFR1-associated death domain protein (TRADD) and Fas-associated protein with death domain (FADD). These adaptor proteins then bind other effector enzymes to trigger various signalling cascades. The DRs may be divided into two categories based on the adaptor protein to which they bind: CD95, DR4 and DR5 bind FADD, while TNFR1 and DR3 bind TRADD. D6 can bind TRADD but its primary physiological adaptor is not yet known.

Activation of TNFR1 by TNF leads to the recruitment of TRADD through a homotypic interaction with the DD of TNFR1. Recruitment of TRADD to TNFR1 occurs within minutes following ligand binding to the extracellular portion of the receptor and leads to two major TNF-induced responses, activation of NF-KB and apoptosis. In more detail, TNFR1 stimulation leads to rapid assembly of complex I, comprising the receptor itself, TRADD, receptor-interacting protein 1 (RIP1), tumour necrosis factor receptor associated factor 2 (TRAF2) or TRAF5 and cellular inhibitor of apoptosis proteins (cIAPs) 1 and 2. TRAF2 is K63-linked poly-ubiquinated upon TNF stimulation, dependent on RING and zinc-finger domain of TRAF2 and requiring the ubiquitin-conjugating enzyme 13 (Ubc13)-ubiquitin-conjugating enzyme E2 variant 1 (Uev1A) complex. RIP1 is also K63-linked poly-ubiquinated, at lysine 377 in its intermediate domain, in response to TNF-dependent association of complex I. Ubiquinated RIP1 then interacts with transforming growth factor-β-activated kinase 1 (TAK1) via TAK1-binding proteins 1 and 2 (TAB1,2). TAK1 in turn activates the IKB kinase (IKK) complex containing IKKα, IKKβ and IKKγ (also known as “NEMO”). IKB, which retains NF-KB in the cytoplasm, is phosphorylated by the IKK complex and is then subjected to polyubiquination at lysine 48 and proteasomal degradation, releasing NF-KB to move into the nucleus and triggering the NF-KB signalling pathway. NF-KB induces transcription of multiple genes, including those encoding proinflammatory cytokines and chemokines, as well as antiapoptotic factors such as cIAPs and c-FLIP. Complex I also activates the MAP kinase JNK and p38, inducing genes that regulate proliferation, differentiation, inflammation or apoptosis:

Formation of complex I is transient and TNFR1 is endocytosed. In some conditions, the TRADD-RIP1-TRAF2 complex dissociates from TNFR1 and the liberated TRADD then recruits caspase-8 through FADD, leading to the formation of the cytoplasmic complex IIA. FADD also recruits c-FLIP which here determines whether capase-8 activation in complex IIA is effected, leading to apoptosis. RIP1 can also associate with FADD to activate caspase-8 and initiate apoptosis downstream of TNFR1 through complex IIB. Within complex IIB, RIP1 is not ubiquinated and TRADD and TRAF2 are not detected. TRADD knockdown enhances formation of complex IIB, suggesting that RIP1 is an alternative apical adaptor for TNFR1.

DR3 is a DD-containing receptor that is upregulated during T cell activation. An endothelial cell-derived TNF-like factor, TL1A (also known as TL1, TNFSF15 and VEG1), is a ligand for DR3 as well as the decoy receptor TR6/DcR3. DR3 expression is inducible by TNF and IL-1α. TL1A induces the formation of a DR3 signalling complex containing DR3, TRADD, TRAF2, and RIP1, and activates the NF-KB and the extracellular signal-regulated kinase (ERK), JNK and p38 mitogen-activated protein kinase pathways. In T cells, TL1A acts as a costimulator that increases IL-2 responsiveness and secretion of proinflammatory cytokines both in vitro and in vivo. Interaction of TL1A with DR3 is considered to promote T cell expansion during an immune response (whereas TR6 has an opposing effect).

Due to its central role in the inflammatory response, TNF and TNF-like molecules have been targeted as a point of intervention in inflammatory diseases. Elevated levels of TNF have also been implicated in many other disorders and disease conditions, including metabolic diseases (for example, diabetes mellitus such as insulin-resistant diabetes mellitus or non-insulin-resistant diabetes mellitus), hyperlipemia, other insulin-resistant diseases, inflammatory diseases [for example, inflammation, dermatitis, atopic dermatitis, hepatitis, nephritis, glomerulonephritis, pancreatitis, psoriasis, gout, Addison's disease, arthritis (for example, rheumatoid arthritis, osteoarthritis, rheumatoid spondylitis, gouty arthritis, synovitis, etc.), inflammatory ocular diseases, inflammatory pulmonary diseases (for example, chronic pneumonia, silicosis, pulmonary sarcoidosis, pulmonary tuberculosis, adult respiratory distress syndrome [ARDS], severe acute respiratory syndrome [SARS], etc.), inflammatory bowel diseases (for example, Crohn's disease, ulcerative colitis, etc.), allergic diseases (for example, allergic dermatitis, allergic rhinitis, etc.), autoimmune disease, autoimmune haemolytic anaemia, systemic lupus erythematosus, rheumatism, Castleman's disease, immune rejection accompanying transplantation (for example, graft versus host reaction, etc.), central nervous system disorders (for example, central neuropathy, cerebrovascular disease such as cerebral haemorrhage and cerebral infarction, head trauma, spinal cord injury, cerebral oedema, multiple sclerosis, etc.), neurodegenerative disease (for example, Alzheimer's disease, Parkinson's disease, amyotrophic lateral sclerosis [ALS], AIDS encephalopathy, etc.), meningitis, Creutzfeldt-Jakob syndrome, respiratory diseases (for example, asthma, chronic obstructive pulmonary disease [COPD], and so forth), cardiovascular diseases (for example, angina, heart failure [such as congestive heart failure, acute heart failure, chronic heart failure, etc.], myocardial infarction [such as acute myocardial infarction, myocardial infarction prognosis, etc.], atrial myxoma, arteriosclerosis, atherosclerosis, hypertension, dialysis-induced hypotension, thrombosis, disseminated intravascular coagulation [DIC], reperfusion injury, restenosis after percutaneous transluminal coronary angioplasty [PICA], and so forth], urinary diseases (for example, renal failure), bone diseases (for example, osteoporosis), cancerous diseases (for example, malignant tumour [such as tumour growth and metastasis, etc.], multiple myeloma, plasma cell leukaemia, carcinoma, and so forth), and infectious diseases (for example, viral infection [such as cytomegalovirus infection, influenza virus infection, herpes virus infection, corona virus infection, etc.], cachexia associated with infections, cachexia caused by AIDS, toxaemia [for example, sepsis, septic shock, endotoxin shock, gram negative bacterial sepsis, toxic shock syndrome, severe acute respiratory syndrome (SARS) accompanying virus infection, etc.], and so forth).

Modulators of TNF and TNF-like molecules are considered to be potentially useful in the treatment of a wide variety of diseases and disorders such as those noted above. For example, the TNF inhibitors infliximab, etanercept and adalimumab have each been approved for the treatment of rheumatoid arthritis, ankylosing spondylitis, psoriasis, and psoriatic arthritis, while some successes have been achieved (at least in animal studies) with the TNF inhibitor dexanabinol (HU-211) against TNF-mediated effects following closed head injury, and with anti-TNF antibodies against Crohn's disease.

Several assays exist in the prior art to allow monitoring of TNF activity, stimulation and/or levels, and hence identification of a TNF modulator (see Mauro et al., 2009, Methods Mol. Bio. 512: 169-207 for review of related methods). For example, cytotoxicity assays are widely used and involve the use of test cells (such as ME180 cells or L929 cells) which are incubated in the presence of actinomycin D and a cytotoxic level of TNF, with or without a putative TNF modulator. In the presence of a TNF inhibitor, the cytotoxic effect of TNF is reduced. In another prior art assay, iodinated TNF in the presence or absence of a putative TNF modulator is fractionated by native polyacrylamide gel electrophoresis. If TNF becomes bound to a TNF modulator, a complex is formed and this will have a different migration pattern compared with unbound TNF.

Further prior art assays for TNF include:
monitoring the trafficking of TNFR1 and the assembly of complex 1, for example using cell fractionation and immunoprecipitation, respectively;
monitoring activation of caspases;
monitoring production of cytokines such as IL-1 or IL-1 cytokine family members;
monitoring post-translational modification and translocation of NF-KB from cytoplasm to nucleus, for example using Western blotting and immunofluorescence, respectively;
monitoring activation of the IKK complex, for example using gel filtration, Western blotting, and ubiquitin and kinase assays;
monitoring phosphorylation and proteolysis of IKBs, for example using Western blotting;
monitoring induction of NF-KB dependent genes, including reporter genes under NF-KB regulation, for example using quantitative real-time (QRT)-polymerase chain reaction (PCR);
monitoring phosphorylation of proteins involved in the NF-KB pathway; and
determining TNF levels using specific anti-TNF antibodies.

The present invention is directed inter alia to alternative methods for monitoring TNF stimulation and identifying TNF modulators.

According to a first aspect of the present invention, there is provided a method of identifying an agent that modulates a signalling pathway mediated by tumour necrosis factor (TNF; which term encompasses members of the TNF ligand superfamily; see below), comprising the steps of: (i) providing a host cell comprising TNF receptor 1 (TNFR1)-associated death domain (TRADD) (or a functional homologue or a functional fragment of TRADD or the homologue) linked to a reporter molecule; (ii) determining at least one cellular characteristic detectable by the reporter molecule in the host cell in the presence of TNF and in the absence of a candidate modulator; (iii) contacting the host cell with the candidate modulator; (iv) determining the at least one cellular characteristic in the presence of the candidate modulator and TNF; and (v) identifying whether the candidate modulator is an agent that modulates a signalling pathway mediated by TNF, in which a change in the at least one cellular characteristic in the presence of said candidate modulator relative to the at least one cellular characteristic in the absence of the candidate modulator identifies the candidate modulator as an agent that modulates a signalling pathway mediated by TNF.

The present method has an advantage over the prior art assays in that it allows identification of modulators which effect TNF signalling at an early stage of TNF-induced activation, including modulators which act on TNF directly or its receptor (for example, allosteric positive or negative modulators). In contrast to the present method which involves the use of the adaptor or accessory protein TRADD which is recruited primarily on activation of a receptor by TNF, many prior art assays as describe above typically detect downstream activities distal to the activation signal. Due to the pleitropic effects of TNF, such prior art methods are not discriminatory.

As elaborated below, we have surprisingly found that monitoring a cellular characteristic detectable by a reporter molecule linked to TRADD (including functional homologues or fragments as described herein) allows such modulators to be detected in a qualitative, semi-quantitative or quantitative assay. The method is suitable for high-throughput screening (HTS) and high-content screening (HCS), or a combination of HTS and HCS, as elaborated below. This is in part because the method of the invention allows monitoring of a target receptor in its native cellular environment.

The method of the present invention may be described as an accessory protein relocation assay (“APRA”) due to its detection of the accessory or adaptor protein TRADD.

According to the invention, the signalling pathway mediated by TNF may be a pathway is which TRADD is involved or implicated, for example as an accessory protein or an adaptor protein.

By using a yeast 2-hybrid screen, Hsu et al. (1995, Cell 81: 495-504) originally isolated cDNAs encoding the 34-kD TRADD protein using a yeast 2-hybrid screen to identify proteins that interact with the DD of TNFR1. The predicted 312-amino acid TRADD protein contains a 111-amino acid DD with sequence similarity to that of TNFR1. Northern blot analysis revealed that the 1.4-kb TRADD mRNA is expressed ubiquitously. In addition to its role in TNFR1 signal transduction, it is now known that TRADD also serves as intermediate in downstream signalling cascade of other receptors such as TNF-receptor family members (DR3, DR4, DR5, DR6), Toll-like receptors (TLR3, TLR4), the retinoic acid inducible gene (RIG)-like helicase, and the interferon-γ receptor.

In the method, the signalling pathway modulated by the agent may be mediated by TNF binding to the receptor TNFR1. Here, the specificity of the method for TNFR1 will allow identification of agents that modulate TNFR1 activity (for example, by blocking the receptor) but not TNFR2 activity, as TRADD has not been implicated in signalling cascades mediated by TNFR2.

The method may further comprise the step of determining the at least one cellular characteristic in the absence of TNF and in the presence or absence of the candidate modulator. This additional step may for example be used as a control step.

The host cell of the method may comprise a recombinant nucleic acid having a first sequence encoding a TRADD polypeptide (or a functional homologue thereof or a functional fragment of either) and a second sequence encoding a reporter molecule, in which the encoded TRADD polypeptide (or the functional homologue or fragment) and the reporter molecule are linked (for example, functionally or structurally linked or joined), for example as a fusion protein.

The host cell may comprise a recombinant nucleic acid having or consisting of the nucleic acid sequence of SEQ ID NO: 15 or SEQ ID NO: 17, or a nucleic acid sequence having at least 60% sequence identity, for example at least 65%, 70%, 75%, 80%, 85%, 90%, 95%, 96%, 97%, 98% or 99% sequence identity, with either of SEQ ID NO: 15 or SEQ ID NO: 17.

The recombinant nucleic acid disclosed herein may encode a fusion protein comprising or consisting of the amino acid sequence of SEQ ID NO: 16 or SEQ ID NO: 18, or a variant thereof having at least 50% sequence identity, for example at least 55%, 60%, 65%, 70%, 75%, 80%, 85%, 90%, 95%, 96%, 97%, 98% or 99% sequence identity, with the fusion protein.

The host cell may comprise a polypeptide having or consisting of the amino acid sequence of SEQ ID NO: 16 or SEQ ID NO: 18, or a variant thereof having at least 50% sequence identity, for example at least 55%, 60%, 65%, 70%, 75%, 80%, 85%, 90%, 95%, 96%, 97%, 98% or 99% sequence identity, with the polypeptide. The polypeptide or variant may be a fusion protein comprising or consisting of TRADD and EYFP (or functional homologues or functional variants thereof).

The host cell may comprise a polypeptide having or consisting of the amino acid sequence of SEQ ID NO: 2, or a variant thereof having at least 50% sequence identity, for example at least 55%, 60%, 65%, 70%, 75%, 80%, 85%, 90%, 95%, 96%, 97%, 98% or 99% sequence identity, with the polypeptide, in which the polypeptide or its variant is linked to a reporter molecule. The polypeptide may comprise or consist of TRADD (or a functional homologue or fragment thereof).

The agent identified by the method may be a negative modulator, for example a negative allosteric modulator (inhibitor), of a signalling pathway mediated by TNF.

The method of the invention may thus be used to identify an agent that may be suitable for treatment of any of the diseases mentioned in the introduction above.

The agent identified by the method may alternatively be a positive modulator, for example a potentiator, of a signalling pathway mediated by TNF.

The method may further comprise the step of isolating and/or purifying a candidate modulator which is identified as an agent that modulates a signalling pathway mediated by TNF.

In another aspect of the invention, there is provided a method of identifying an agent that modulates binding of TRADD (or a functional homologue or a functional fragment of TRADD or the homologue) to an associated receptor, comprising the steps of: (i) providing a host cell comprising TRADD (or the functional homologue or fragment) linked (for example, functionally or structurally linked or joined) to a reporter molecule; (ii) determining at least one cellular characteristic detectable by the reporter molecule in the host cell using the reporter molecule in the absence of a candidate modulator; (iii) contacting the host cell with the candidate modulator; (iv) determining the at least one cellular characteristic in the presence of the candidate modulator; and (v) identifying whether the candidate modulator is an agent that modulates binding of TRADD (or the functional homologue or fragment) to the associated receptor, in which a change in the at least one cellular characteristic in the presence of said candidate modulator relative to the at least one cellular characteristic in the absence of the candidate modulator identifies the candidate modulator as an agent that modulates binding of TRADD to the associated receptor.

In the method, the host cell may be as disclosed herein.

The associated receptor may be a receptor which is associated with (for example, forms a complex with) TRADD, such as when the receptor is activated by TNF.

The associated receptor may be TNFR1.

Alternatively, the associated receptor may be any one or more of the group consisting of DR3 [SEQ ID NO: 20], DR4 (also known as TNF receptor superfamily member 10A or TRAIL-R1; see for example NCBI Reference Sequence: NP_{—}003835.3), DR5 (also known as TNF receptor superfamily member 10B, TRAIL-R2, TRICK2A, TRICKB, KILLER or Apo2; see for example, GenBank Accession No. BAA33723.1), DR6 (for example, GenBank Accession No. AAC34583.1), TLR3 (for example, GenBank Accession No. U88879.1), TLR4 (for example, NCBI Reference Sequence NM_{—}138554.3), RIG-like helicase, interferon-γ receptor (for example, GenBank Accession No. AAA52731.1), or any receptor associated with TRADD. Further applicable associated receptors may be members of the TNF receptor superfamily (see for example as mentioned in Table 2 of MacEwan, 2002, Br. J. Pharmacol. 135: 855-875).

The associated receptor may be endogenous to the host cell. Alternatively, the associated receptor may be heterologous and/or introduced into the host cell.

The method may be conducted in the presence or absence of a signalling molecule that interacts with the associated receptor.

The method may further comprise the step of isolating and/or purifying a candidate modulator which is identified as an agent that modulates binding of TRADD to an associated receptor.

Step (ii) of the methods of the invention may be performed prior to, after, or simultaneously with steps (iii) and (iv) to allow identification of a change in the at least one cellular characteristic in step (v). For example, the steps may be performed in wells of a multiwell plate.

The at least one cellular characteristic according to the methods of the invention may be cellular positioning (for example, intracellular positioning) of TRADD or its functional homologue or fragment. Alternatively, the at least one characteristic may be distribution, environment and/or activity of TRADD or its functional homologue or fragment. The at least one cellular characteristic may additionally or alternatively be localisation, distribution, structure or activity of TRADD or its functional homologue or fragment.

The at least one characteristic (such as for example intracellular positioning) according to the methods of the invention may be determined in step (iv) of the methods up to 60 minutes after both the candidate modulator and either TNF or the signalling molecule that interacts with the associated receptor are present in the host cell, for example between 1 and 60 minutes, between 5 and 30 minutes, or between 10 and 15 minutes after both are present. Typically, in step (iv) of the methods, the host cell may be incubated first with the candidate modulator and then either TNF or the signalling molecule that interacts with the associated receptor added.

The ability to identify an agent according to the methods with the time frame indicated above is a significant advantage over related prior art methods which may require at least 24 h to achieve a result.

The at least one characteristic may be determined on live or fixed cells. Fixed cells are cells which have been processed, for example by cross-linking, precipitation or freezing, to retain their structural organization for subsequent staining and visualisation. The cells may be fixed by cross-linking by treatment with reagents such as aldehydes (for example, formaldehyde and glutaraldehyde) that penetrate into the cells and form covalent cross-links between intracellular components.

The reporter molecule for use in the methods of the inventions may be a label, for example a mass tag, biotin, an enzyme, and/or a nanoparticle.

The reporter molecule for use in the methods may be a fluorescent reporter molecule, for example a fluorescent protein (FP) such a green fluorescent protein (GFP), yellow fluorescent protein (YFP) or enhanced YFP (EYFP), a luciferase, or a functional homologue or fragment of any of these.

The reporter molecule may be a fluorophore, for example one which is detectable by a fluorescence detector. Here, the at least one cellular characteristic may be determined using fluorescence microscopy.

The reporter molecule of the invention may comprise a fluorescence moiety. The fluorescence signal from the fluorescence moiety may be used to detect the at least one cellular characteristic. The fluorescence signal may be converted into digital data to measure (for example quantitatively measure) the at least one cellular characteristic.

An automated microscope system may be used in the method. Such a system may allow one or more or all of the following: high content screening; analysis of host cells containing reporter molecules (for example, fluorescent reporter molecules) in an array of locations; treating the host cells in the array of locations with one or more candidate modulators; imaging numerous cells in each location, for example with fluorescence optics; converting the optical information into digital data; utilising the digital data to determine the location (or position), distribution, environment and/or activity of the reporter molecules in the cells and/or the distribution of the cells; and interpreting that information in terms of a positive, negative or null effect of the candidate modulator on the at least one cellular characteristic.

The host cell may for example be a mammalian cell such as for example a cell of bovine, porcine, rodent, monkey or human origin. The mammalian cell may for example be any one of the group consisting of a HeLa cell, a U2OS cell, a Chinese hamster ovary (CHO) cell, a CHO-KL cell, a HEK293 cell, a HEK293T cell, an NSO cell, a CV-1 cell, an L-M(TK-) cell, an L-M cell, a Saos-2 cell, a 293-T cell, a BCP-1 cell, a Raji cell, an NIH/3T3 cell, a C127I cell, a BS-C-1 cell, an MRC-5 cell, a T2 cell, a C3H10T1/2 cell, a CPAE cell, a BHK-21 cell, a COS cell (for example, a COS-1 cell or a COS-7 cell), a Hep G2 cell, and an A-549 cell. Such cells and other suitable cells are publicly available, for example from commercial sources such as the American Type Culture Collection (ATCC), the European Collection of Cell Cultures (ECACC) and/or the Riken Cell Bank (Tokyo, Japan).

The method of the invention may comprise the step of imaging or scanning multiple host cells.

The invention also encompasses a novel agent that modulates a signalling pathway mediated by TNF, in which the agent is detected according to the method disclosed herein. A method of treating a disease regulated by TNF using the agent is also encompassed.

Further encompassed is a novel agent that modulates binding of TRADD to an associated receptor, in which the agent is detected according to the method disclosed herein. A method of treating a disease regulated by a molecule that interacts with TRADD and/or the associated receptor using the agent is also encompassed.

According to another aspect of the invention, there is provided an isolated nucleic acid comprising a first sequence encoding a TRADD polypeptide (or a functional homologue of the polypeptide or a functional fragment of the polypeptide and/or the homologue) and a second sequence encoding a reporter molecule, in which the encoded TRADD polypeptide (or the functional homologue or fragment) and the reporter molecule are linked (for example, functionally or structurally linked or joined), for example as a fusion protein.

The isolated nucleic acid has been found, as shown herein, to be useful for example in the methods disclosed herein.

The reporter molecule is a fluorescent reporter molecule, for example an FP such a GFP, YFP or EYFP, a luciferase, or a functional homologue or functional fragment of any of these.

The nucleic acid of the invention may comprise or consist of the nucleic acid sequence of SEQ ID NO: 15 or SEQ ID NO: 17, or a nucleic acid sequence having at least 60% sequence identity, for example at least 65%, 70%, 75%, 80%, 85%, 90%, 95%, 96%, 97%, 98% or 99% sequence identity, with either of SEQ ID NO: 15 or SEQ ID NO: 17.

The nucleic acid may encode a fusion protein comprising or consisting of the amino acid sequence of SEQ ID NO: 16 or SEQ ID NO: 18, or a variant thereof having at least 50% sequence identity, for example at least 55%, 60%, 65%, 70%, 75%, 80%, 85%, 90%, 95%, 96%, 97%, 98% or 99% sequence identity, with the fusion protein.

The nucleic acid may further comprise a promoter sequence which controls expression of the first and second sequences. The promoter sequence may allow inducible or constitutive expression of the first and second sequences.

The nucleic acid may be suitable for use in a method of detecting an agent that modulates a signalling pathway mediated by TNF.

The nucleic acid may be suitable for use in a method of detecting TRADD modulators, for example modulators that affect TRADD (or functional homologues or fragments) binding to TNFR1.

Also provided according to the invention is a polypeptide comprising or consisting of the amino acid sequence of SEQ ID NO: 16 or SEQ ID NO: 18, or a variant thereof having at least 50% sequence identity, for example at least 55%, 60%, 65%, 70%, 75%, 80%, 85%, 90%, 95%, 96%, 97%, 98% or 99% sequence identity, with the polypeptide.

The polypeptide or variant may be a fusion protein comprising or consisting of TRADD and EYFP (or a functional homologue of TRADD and/or EYFP, or a functional fragment of TRADD and/or EYFP, or a functional fragment of either or both homologue).

Further provided according to the invention is a polypeptide comprising or consisting of the amino acid sequence of SEQ ID NO: 2, or a variant thereof having at least 50% sequence identity, for example at least 55%, 60%, 65%, 70%, 75%, 80%, 85%, 90%, 95%, 96%, 97%, 98% or 99% sequence identity, with the polypeptide, in which the polypeptide or its variant is linked (for example, tagged, functionally or structurally linked, or functionally or structurally joined) to a reporter molecule.

The polypeptide or variant may comprise or consist of TRADD.

Here, the reporter molecule may be a non-protein molecule. For example, the polypeptide or TRADD or their variants may be labelled with a detectable reporter molecule.

The reporter molecule may be covalently or non-covalently bound to the polypeptide or TRADD or their variants.

Further provided accorded to the invention is an expression vector comprising a nucleic acid as disclosed herein.

Expression of the nucleic acid may be driven by a constitutive or inducible promoter. Typically, the promoter is positioned upstream of the nucleic acid to allow transient or stable expression, for example in mammalian cells.

The expression vector may comprise a Tet-ON® inducible expression system.

Use of an inducible expression system allows higher levels of the polypeptide of the invention to be present when desired or required.

Expression may be inducible for example upon addition of doxycyclin, tetracycline, or an analogue of either, such as in a mammalian cell for example a HeLa cell or other cells disclosed herein.

In another aspect of the invention, there is provided a host cell comprising a nucleic acid as disclosed herein, a polypeptide as disclosed herein, or an expression vector as disclosed herein.

The nucleic acid, expression vector or polypeptide may be transiently or stably transfected into the host cell.

The host cell may for example be a mammalian cell, such as those disclosed herein.

The invention also encompasses a kit comprising a nucleic acid as disclosed herein, a polypeptide as disclosed herein, an expression vector as disclosed herein, or a host cell as disclosed herein, for use in a method as disclosed herein.

The invention may be used for high-throughput screening (HTS). According to a further aspect of the invention, there is provided a HTS assay using a method as disclosed herein, a nucleic acid as disclosed herein, a polypeptide as disclosed herein, an expression vector as disclosed herein, or a host cell as disclosed herein.

The invention may also be used for high-content screening (HCS). HCS addresses the need for more detailed information concerning the temporal-spatial dynamics of cell constituents and processes. HCS as used herein automates the extraction of fluorescence information (typically multicolour fluorescence information) derived from specific detectable fluorescence-based reagents incorporated into cells. These cells may be analysed using an optical system that can measure spatial and optionally temporal dynamics. HCS systems and methods as disclosed in EP1214980 are suitable for the present invention. Particularly suitable are the Image Acquisition and Analysis features disclosed in EP1214980.

According to a further aspect of the invention, there is provided a HCS assay using a method as disclosed herein, a nucleic acid as disclosed herein, a polypeptide as disclosed herein, an expression vector as disclosed herein, or a host cell as disclosed herein.

Also provided is the use of the present invention in a system which combines HTS and HCS. Such combination systems may, for example, be as disclosed in EP1214980. Particularly suitable are the Image Acquisition and Analysis features disclosed in EP1214980.

As used herein, the term “modulator” refers generically to a positive or a negative modulator. A positive modulator may be a positive allosteric modulator (enhancer). A negative modulator, also referred to herein as an inhibitor, may be a negative allosteric modulator (inhibitor).

As used herein, the term “reporter molecule” encompasses functional homologues and functional fragments of known reporter molecules which maintain functionality as a reporter molecule. The term also encompasses non-protein reporter molecules where applicable, as described herein.

As used herein, the term “linked” means that the TRADD polypeptide (or its functional homologue or fragment) and reporter molecules are structurally or functionally linked or joined. To obtain a fusion protein (an example of structurally linked encoded sequences), an open reading frame DNA sequence encoding the TRADD polypeptide (or its functional homologue or fragment) may be joined with a second open reading frame DNA sequence encoding the reporter molecule such as for example EYFP.

As used herein, a host cell “comprising” a protein or polypeptide (such as TRADD [or its functional homologue or fragment] linked to a reporter molecule) means that the host cell includes the polypeptide, for example following in vivo expression from an expression vector of the type disclosed herein. The polypeptide will preferably be found in a form suitable for detection using the reporter molecule. Typically, the polypeptide will be intracellular.

The term “tumour necrosis factor” (TNF) as used herein includes the known synonyms TNFα, cachectin, TNFSF2 and differentiation inducing factor (DIF). Homologues of TNF (see for example as mentioned in Table 1 in MacEwan, 2002, Br. J. Pharmacol. 135: 855-875) are in one aspect also encompassed by the invention. Homologues of TNF (also referred to as the “TNF ligand superfamily” or “TNF-like” molecules) encompassed by the term “TNF” as used herein include 4-1BB ligand (also known as 4-1BBL and TNFSF9), APRIL (also known as TALL2 and TNFSF13), CD27 ligand (also known as CD27L, CD70 and TNFSF7), CD30 ligand (also known as CD30L and TNFSF8), CD40 ligand (also known as CD40L, CD154, GP39, HIGM1, IMD3, TNFSF5 and TRAP), Fas ligand (also known as APT1 LG1, FasL and TNFSF6), GITR ligand (also known as AITRL, GITRL, TL6 and TNFSF18), LIGHT (also known as HVEM ligand, TL1 and TNFSF14), LTa (also known as LT, TNTB, TNFSF1), LTb (also known as TNFC, TNFSF3 and p33), OX40 ligand (also known as gp34, OX40L, TNFSF4 and TXGP1), RANK ligand (also known as ODF, OPGL, RANKL, TNFSF11 and TRANCE), THANK (also known as BAFF, BLYS, TALL1 and TNFSF13B), TRAIL (also known Apo2 ligand, TL2 and TNFSF10), TWEAK (also known as Apo3 ligand, DR3L and TNFSF12), and VEG1 (also known as TL1, TL1A and TNFSF15).

As used herein, the “absence” of the candidate modulator and/or TNF and/or the signalling molecule that interacts with the associated receptor in one aspect does not exclude the presence of these substances in low or residual levels in the host cell. The methods of the invention may be performed using higher and lower concentrations of these substances—rather than presence and absence, respectively—to ascertain in step (v) whether there has been a change in the at least one characteristic.

Sequence identity between nucleotide or amino acid sequences can be determined by comparing an alignment of the sequences, for example using the entire length of the sequences of the present invention. When an equivalent position in the compared sequences is occupied by the same base or amino acid, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62. Default parameters for nucleotide sequence comparisons (“DNA Molecule” option) may be Gap Extend penalty: 0.5, Gap Open penalty: 10.0, Matrix: DNAfull.

Variants of the nucleic acids and polypeptides of the inventions are also encompassed. The term “variant” in relation to a nucleic acid sequence means any substitution of, variation of, modification of, replacement of deletion of, or addition of one or more nucleic acid(s) from or to a polynucleotide sequence providing the resultant peptide sequence encoded by the polynucleotide exhibits at least the same properties as the peptide encoded by the basic sequence. The term therefore includes allelic variants and also includes a polynucleotide which substantially hybridises to the polynucleotide sequences of the present invention. Such hybridisation may occur at or between low and high stringency conditions, each of which conditions are encompassed by the invention. In general terms, low stringency conditions can be defined a hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tₘ of the probe (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (1989; Molecular Cloning, Cold Spring Harbor Laboratory Press, Cold Spring Harbor).

The polypeptides of the invention may employ amino acid analogs, which are defined as any of the amino acid-like compounds that are similar in structure and/or overall shape to one or more of the twenty L-amino acids commonly found in naturally occurring proteins. These twenty L-amino acids are defined and listed in WIPO Standard ST. 25 (1998), Appendix 2, Table 3 as alanine (Ala or A), cysteine (Cys or C), aspartic acid (Asp or D), phenylalanine (Phe or F), glutamate (Glu or E), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y).

An amino acid analog may thus include natural amino acids with modified side chains or backbones. The analogs may share backbone structures, and/or even the most side chain structures of one or more natural amino acids, with the only difference(s) being containing one or more modified groups in the molecule. Such modification may include substitution of an atom (such as N) for a related atom (such as S), addition of a group (such as Methyl, or hydroxyl group, etc.) or an atom (such as Cl or Br, etc.), deletion of a group (supra), substitution of a covalent bond (single bond for double bond, etc.), or combinations thereof. Amino acid analogs may include α-hydroxy acids, and β-amino acids, and can also be referred to as “modified amino acids”. Amino acid analogs may either be naturally occurring or unnaturally occurring (e.g. synthesized). As will be appreciated by those skilled in the art, any structure for which a set of rotamers is known or can be generated can be used as an amino acid analog. The side chains may be in either the (R) or the (S) configuration (or D- or L-configuration).

Further features and particular non-limiting embodiments of the present invention will now be described below with reference to the following drawings, in which:

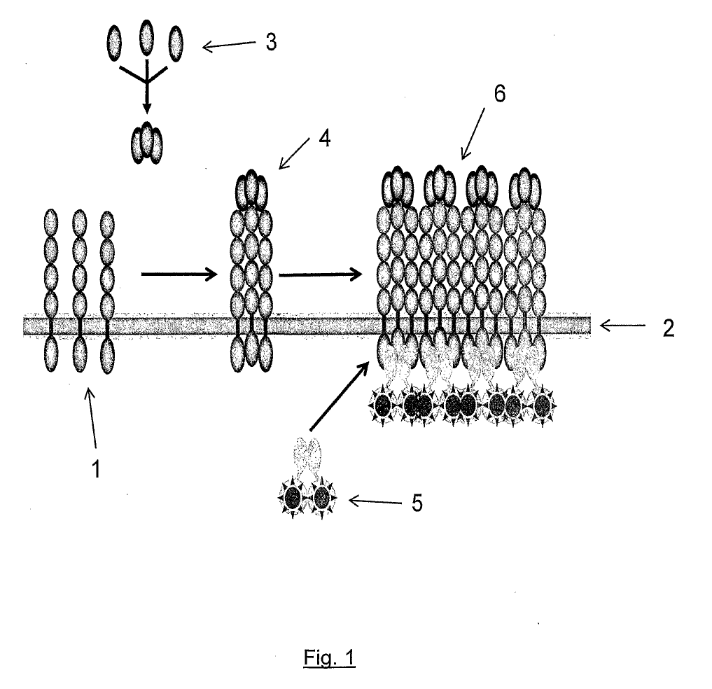

FIG. 1 is a schematic illustration of an embodiment of the method according to the invention;

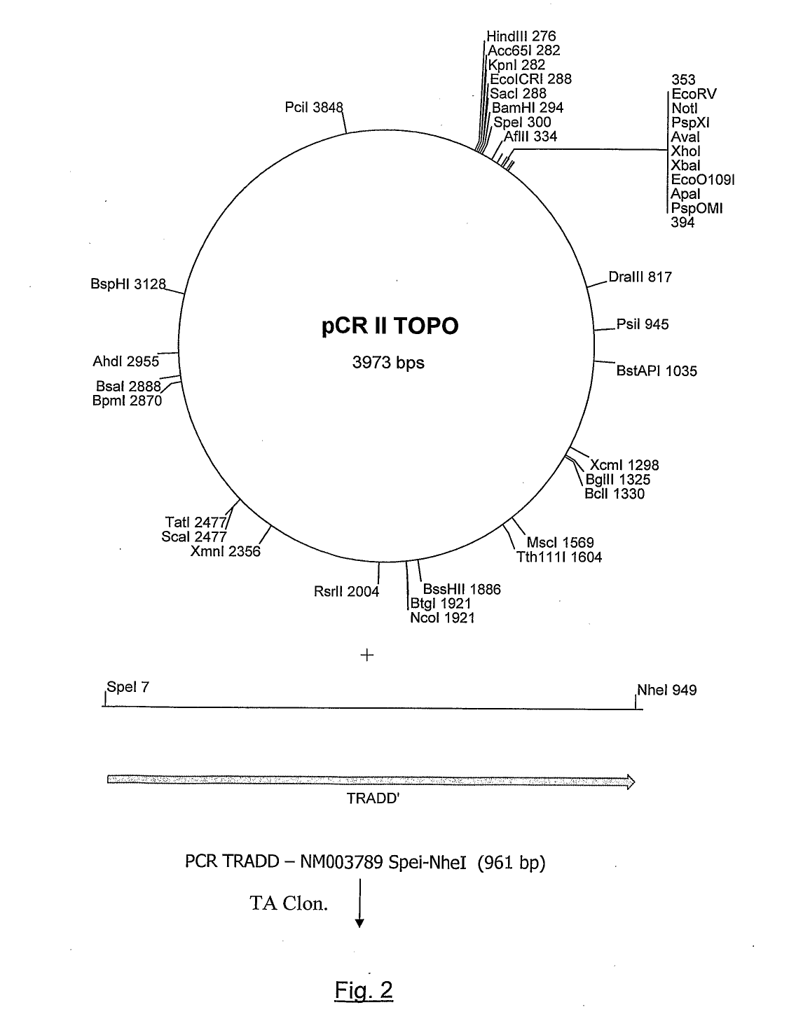

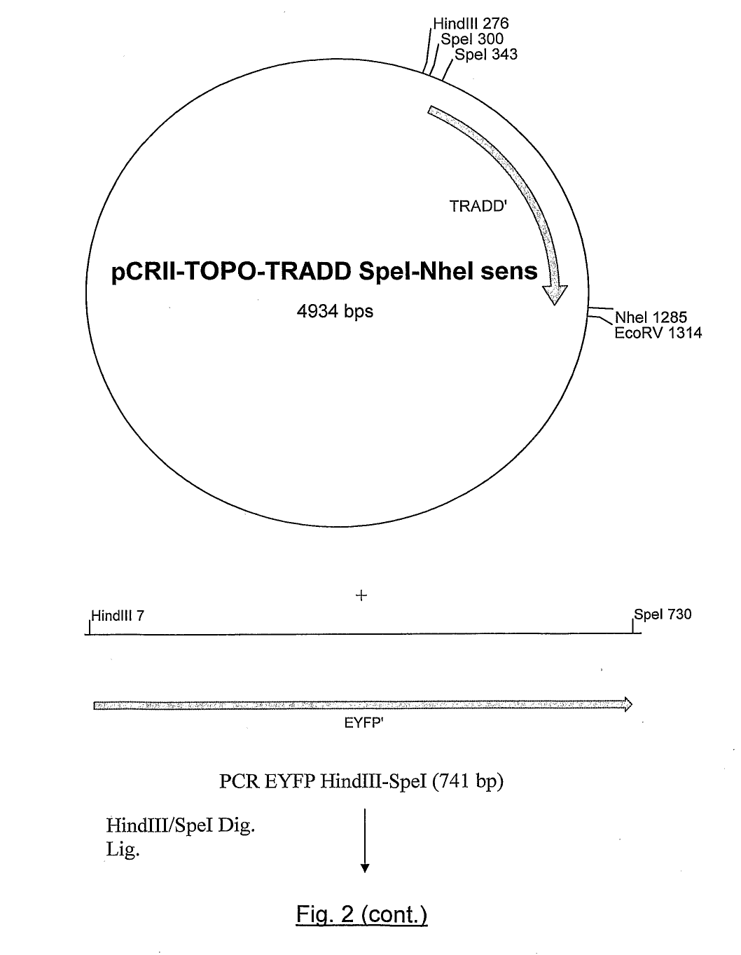

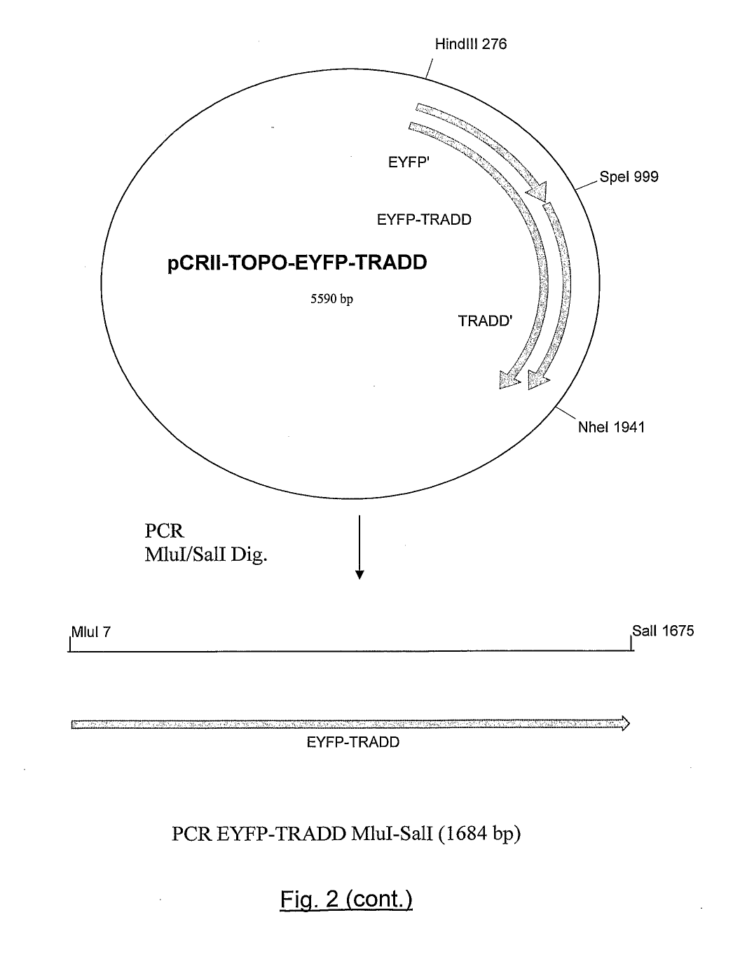

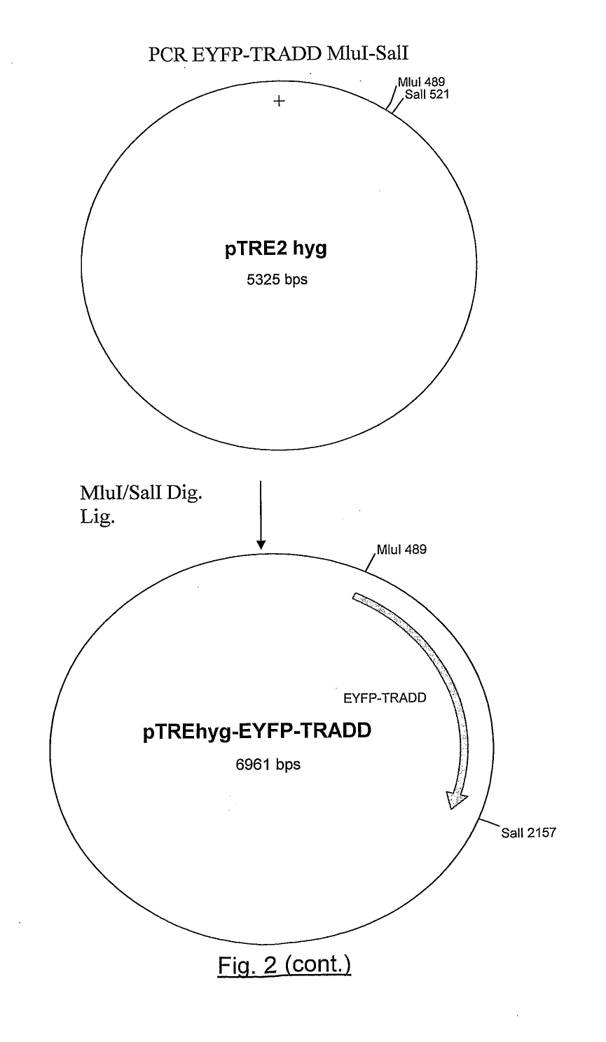

FIG. 2 shows a cloning strategy used to produce a pTREhyg-EYFP-TRADD expression vector allowing expression in a mammalian cell of the EYFP-TRADD fusion protein (SEQ ID NO: 16). In the figure, “TA Clon.” refers to TA cloning, “Dig.” refers to digestion with the indicated restriction endonuclease(s), and “Lig.” refers to ligation;

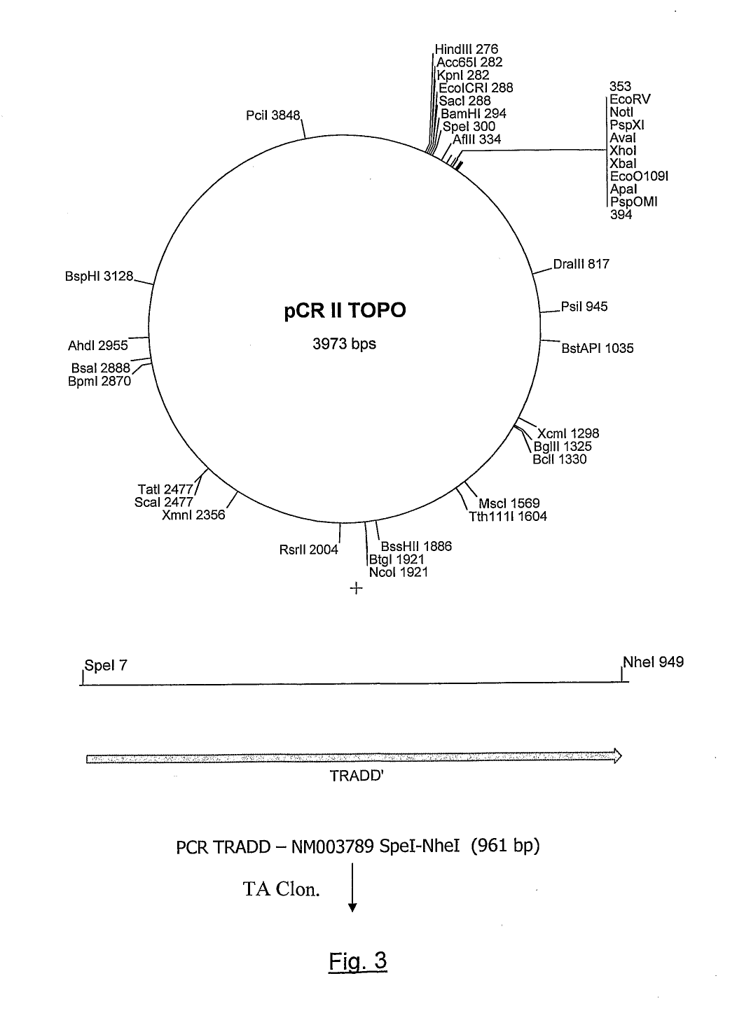

FIG. 3 shows a cloning strategy used to produce a pTREhyg-TRADD-EYFP expression vector allowing expression in a mammalian cell of the TRADD-EYFP fusion protein (SEQ ID NO: 18). In the figure, “TA Clon.” refers to TA cloning, “Dig.” refers to digestion with the indicated restriction endonuclease(s), and “Lig.” refers to ligation;

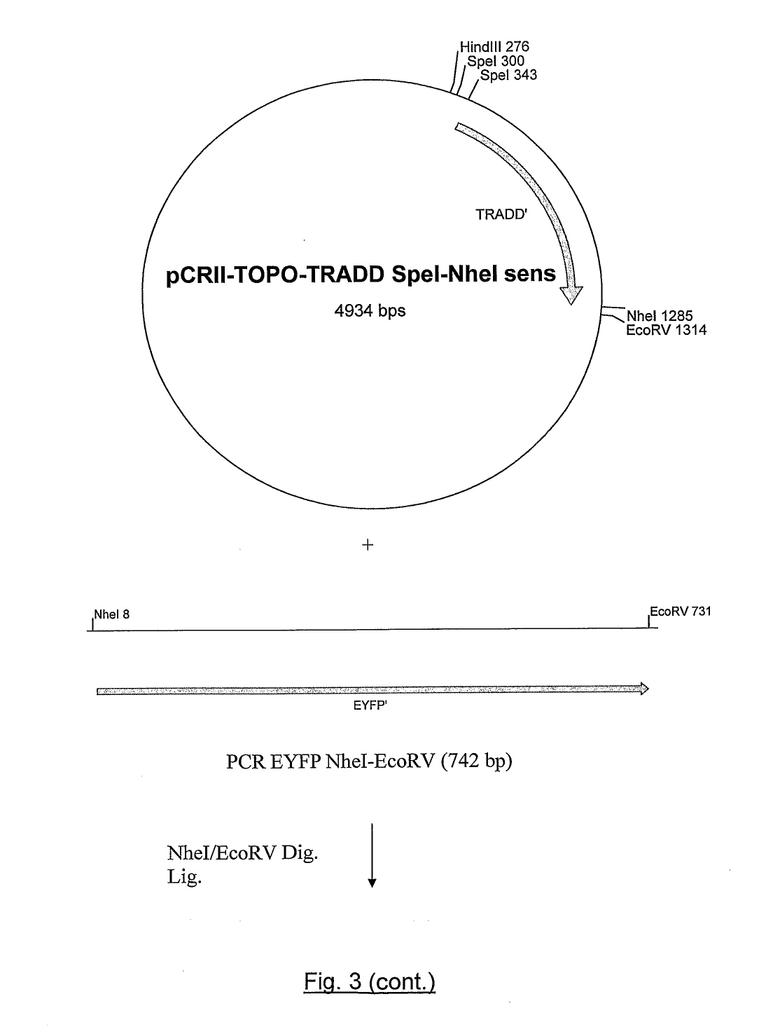

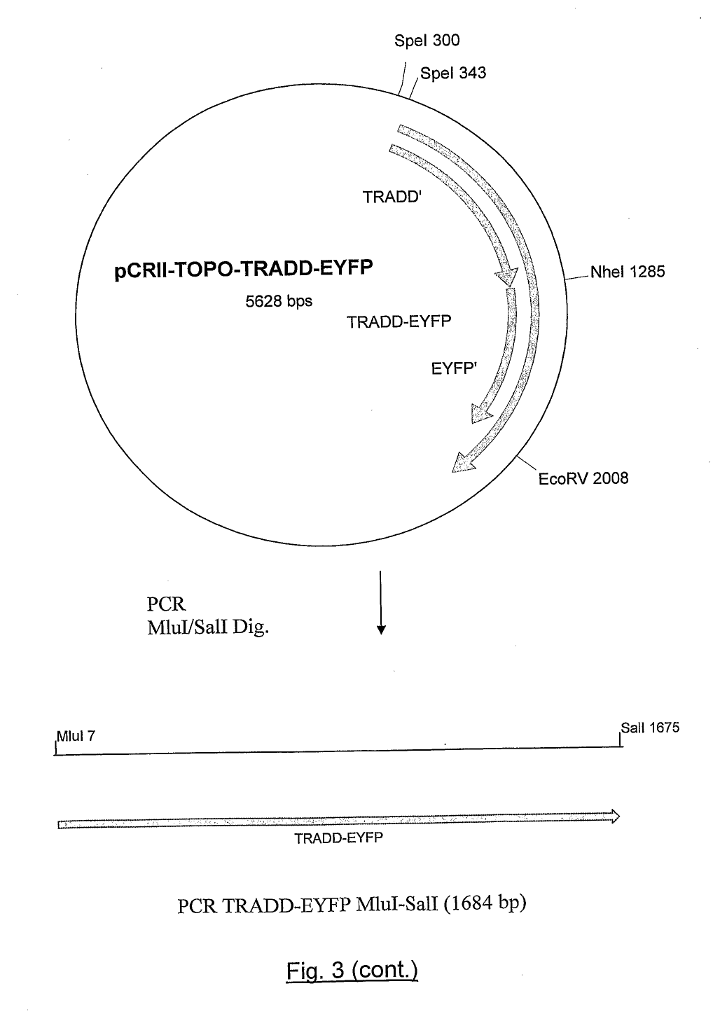

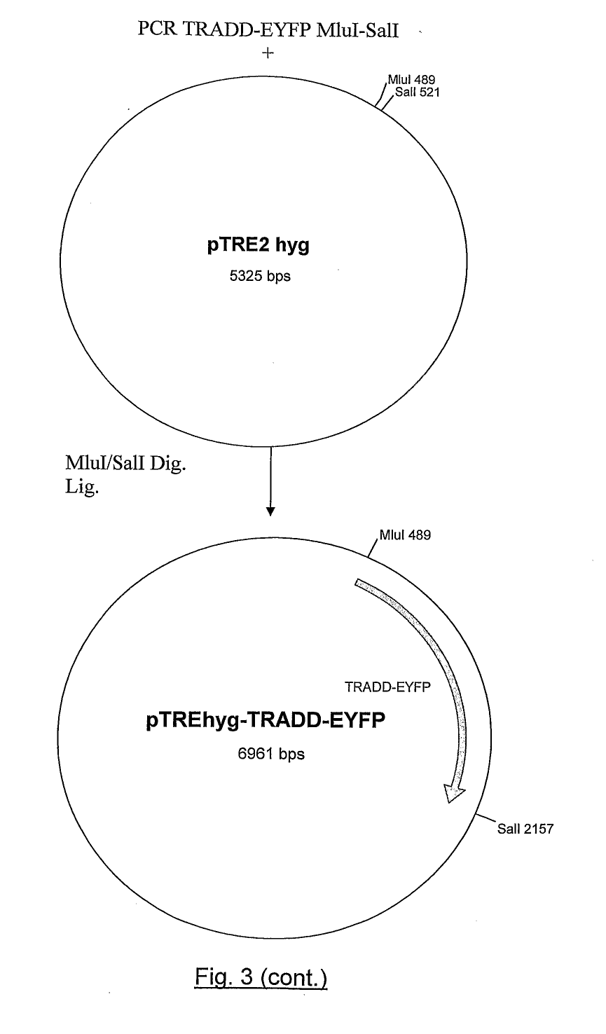

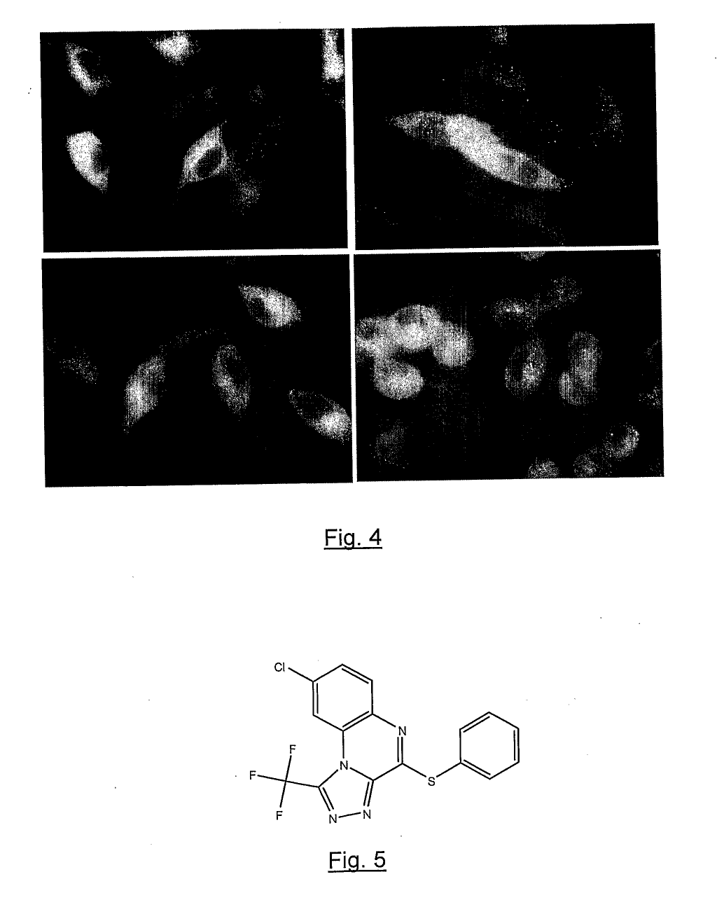

FIG. 4 shows fluorescence detection of TRADD-EYFP (clone #49; top panels) or EYFP-TRADD (clone #6; lower panels) in untreated (control) HeLa cells (left panels) and in HeLa cells with 1000 ng/ml TNF (right panels);

FIG. 5 shows the chemical structure of a prior art TNF antagonist R-7050 (Guraraja et al., 2007, Chem. Biol. 14: 1105-1118);

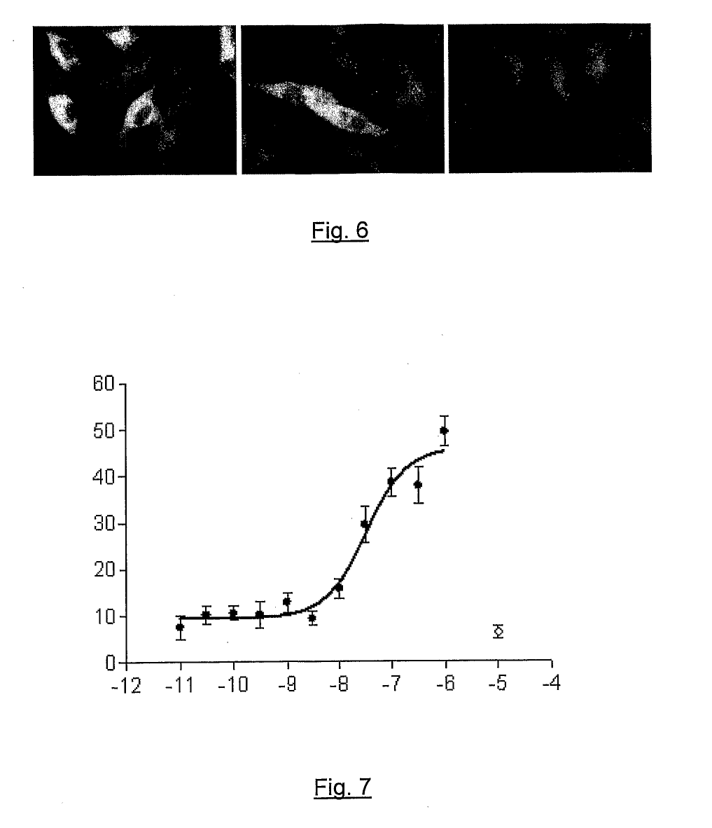

FIG. 6 shows fluorescence detection of TRADD-EYFP in untreated HeLa cells (left panel), HeLa cells with 1000 ng/ml TNF (middle panel), and HeLa cells with 1000 ng/ml TNF and 100 μM R-7050 (right panel). All cells were from clone HeLa/TRADD-EYFP#49;

FIG. 7 is a graph showing a dose-response curve of TNF-mediated relocation of TRADD-EYFP in HeLa cells (clone HeLa/TRADD-EYFP#49). The x-axis shows Log [TNF] in g/ml, while the y-axis shows % Responder (spot count per object). TNF values are plotted as “▪”, while background cell culture medium is shown as “∘”;

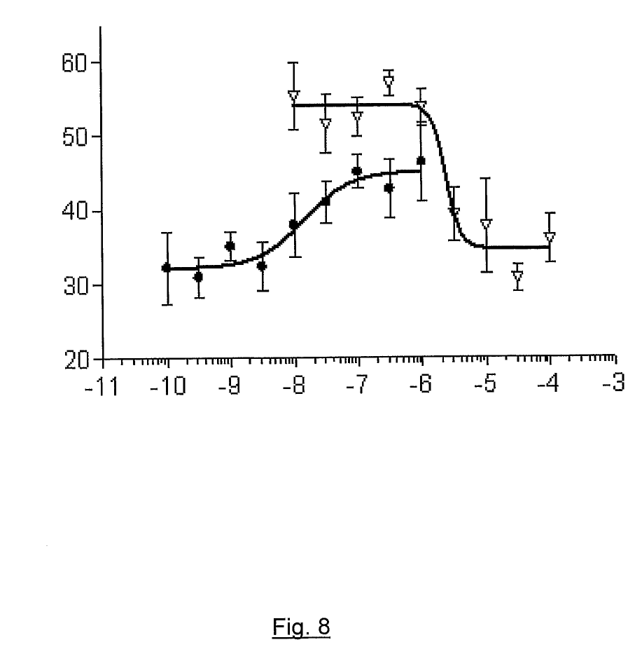

FIG. 8 is a graph showing a dose-response curve of inhibition by R-7050 of TNF-mediated relocation of TRADD-EYFP in HeLa cells (clone HeLa/TRADD-EYFP#49). The x-axis shows Log [TNF] in g/ml or Log [R-7050] in M, while the y-axis shows spots count per cell % Responder (spot count per object). TNF values are plotted as “”, while R-7050 values are plotted as “∇”;

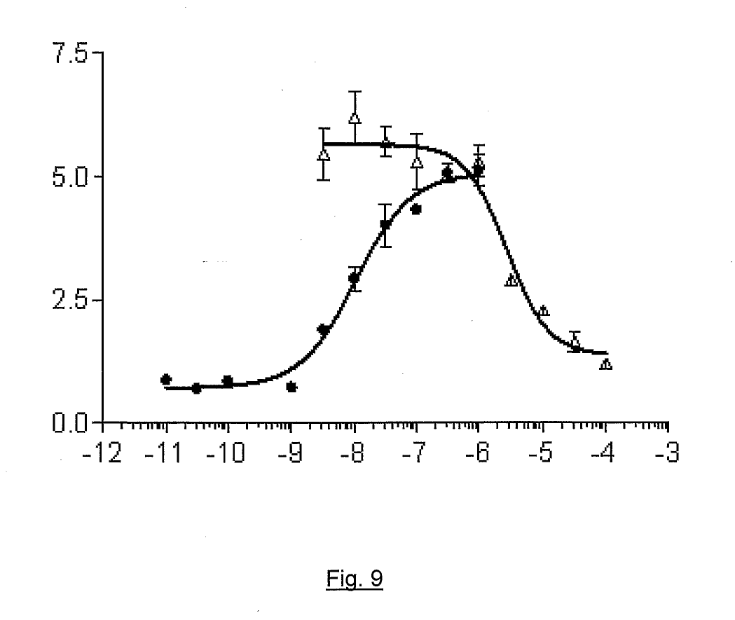

FIG. 9 is a graph showing inhibition by etanercept (Enbrel®) of TNF-mediated relocation of TRADD-EYFP in HeLa cells (clone HeLa-Tet/TRADD-EYFP#49). TNF values are plotted as “”, while etanercept values are plotted as “Δ”;

FIG. 10 is a scatter plot showing TRADD-EYFP fluorescence spots per cell measured in 384-well setting on clone HeLa-Tet/TRADD-EYFP#49 cells treated with TNF to show TNF-induced relocation, in the presence and absence of etanercept. The y-axis shows spot count per cell, while the x-axis represents the following cells—(A) 300 ng/ml TNF, (B) 300 ng/ml TNF and etanercept, (C) background milieu, (D) 300 ng/ml TNF (repeat of A), and (E) TNF PLAQUE (combined cells with 300 ng/ml TNF);

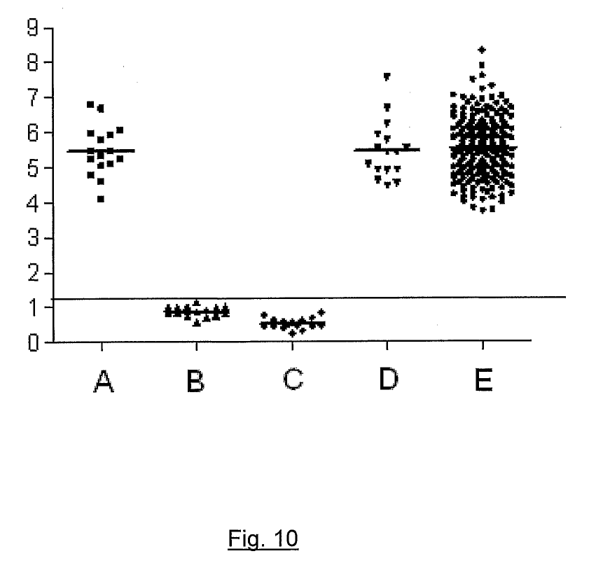

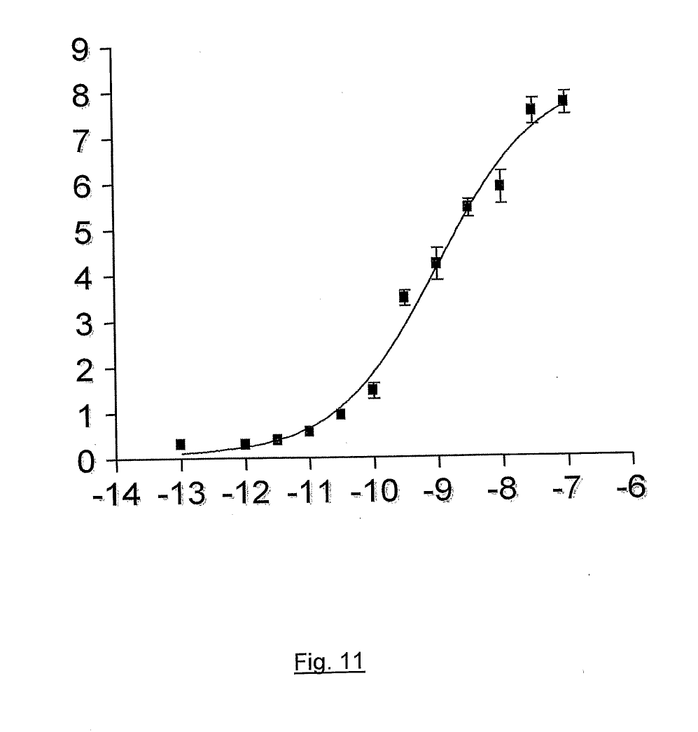

FIG. 11 is a graph showing a dose-response “performance” curve of an embodiment of the invention using TNF-mediated relocation of TRADD-EYFP in Clone #49-E2 HeLa cells. The x-axis shows Log [TNF] in g/ml, while the y-axis shows spots count per cell;

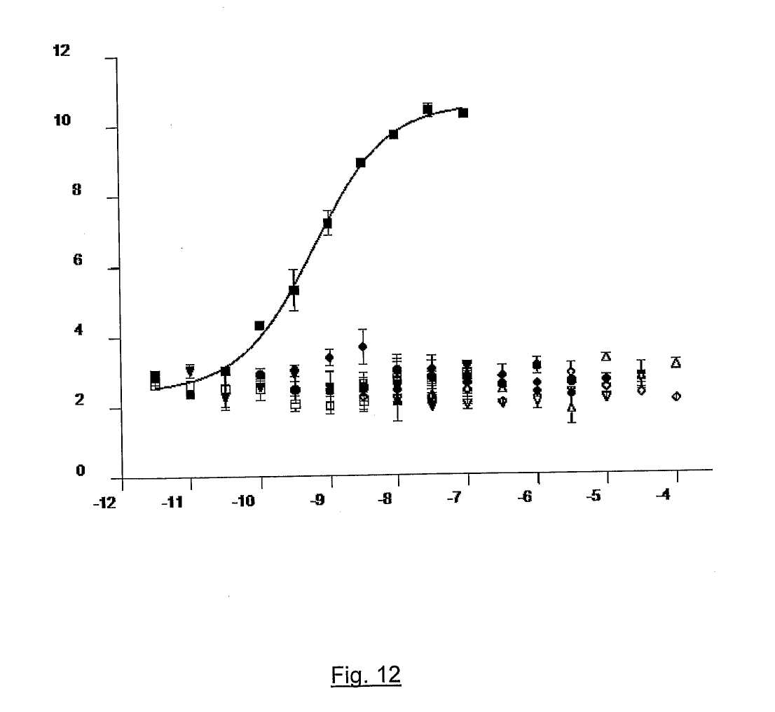

FIG. 12 is a graph showing the selectivity of the embodiment depicted in FIG. 11. The x-axis shows Log [compound] in g/ml, while the y-axis shows spots count per cell. The compounds/molecules tested were TNF (▪), TL1A (▾), IL1β (), CD40L (▪), TRAIL (□), anti-FAS (Δ), polyinosinic-polycytidylic acid (Poly I:C) (∇) and lipopolysaccharides (LPS) (∘);

FIG. 13 is a graph showing the inhibition of the embodiment depicted in FIG. 11 by etanercept. The x-axis shows Log [etanercept] in g/ml, while the y-axis shows % inhibition;

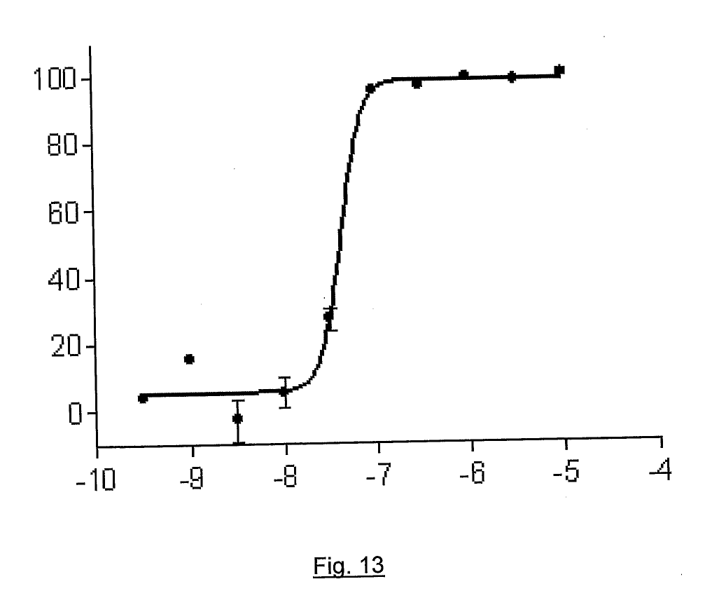

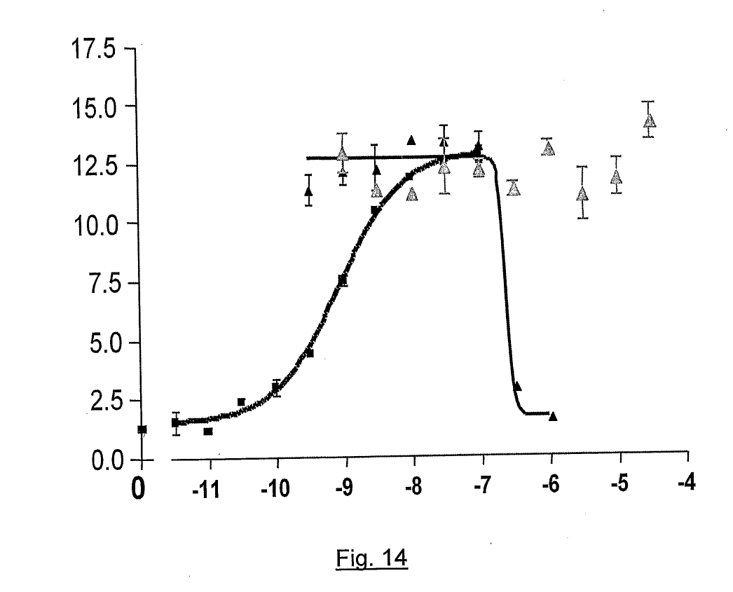

FIG. 14 is a graph showing the inhibition of the embodiment depicted in FIG. 11 by the caspase-8 inhibitor Z-IETD-FMK. Comparative TNF and etanercept values are also provided. The x-axis shows Log [compound] in M or g/ml, while the y-axis shows average spots/object. The compounds depicted are TNF (▪), etanercept (▴) and Z-IETD-FMK (Δ);

FIG. 15 is a graph showing the inhibition of the embodiment depicted in FIG. 11 by the IKK inhibitor IKK16. Comparative TNF and etanercept values are also provided. The x-axis shows Log [compound] in M or g/ml, while the y-axis shows average spots/object. The compounds depicted are TNF (▪), etanercept (♦) and IKK16 (Δ);

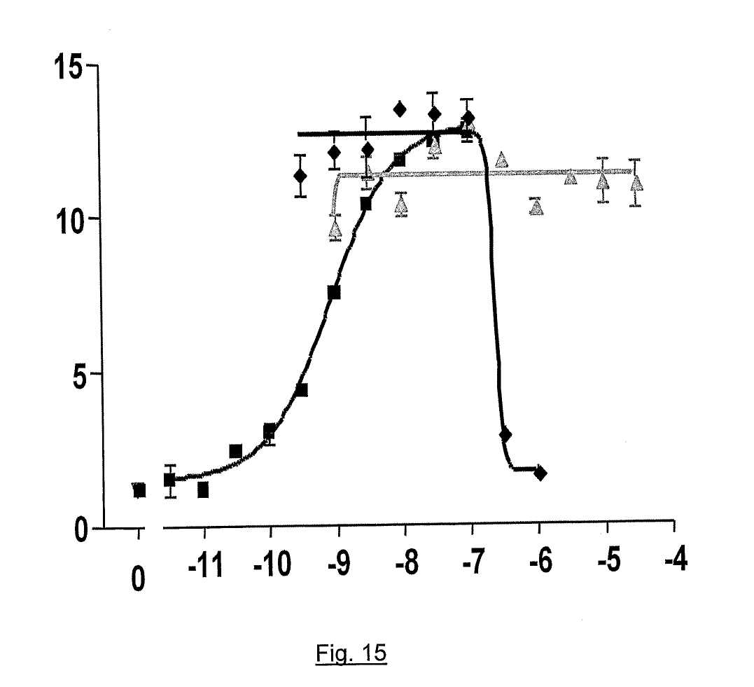

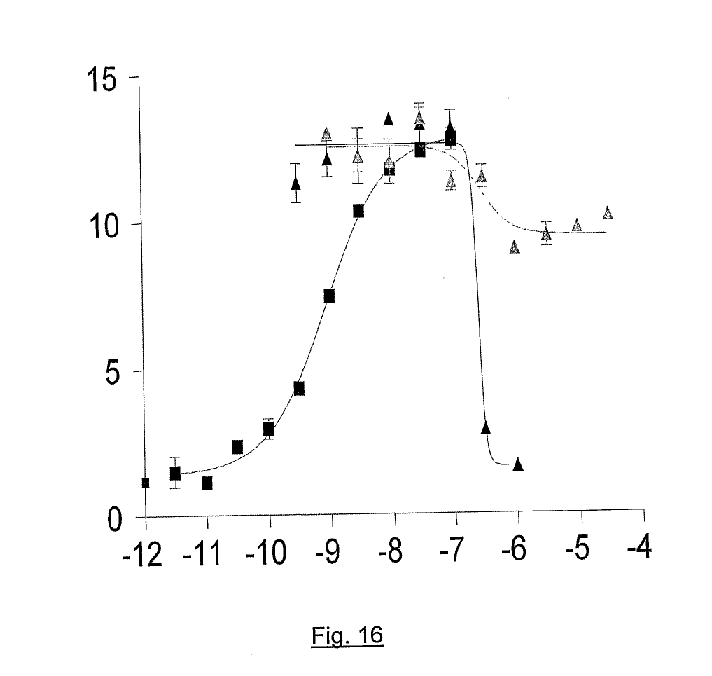

FIG. 16 is a graph showing the inhibition of the embodiment depicted in FIG. 11 by the actin polymerisation inhibitor cytochalasin D. Comparative TNF and etanercept values are also provided. The x-axis shows Log [compound] in M or g/ml, while the y-axis shows average spots/object. The compounds depicted are TNF (▪), etanercept (▴) and cytochalasin D (Δ);

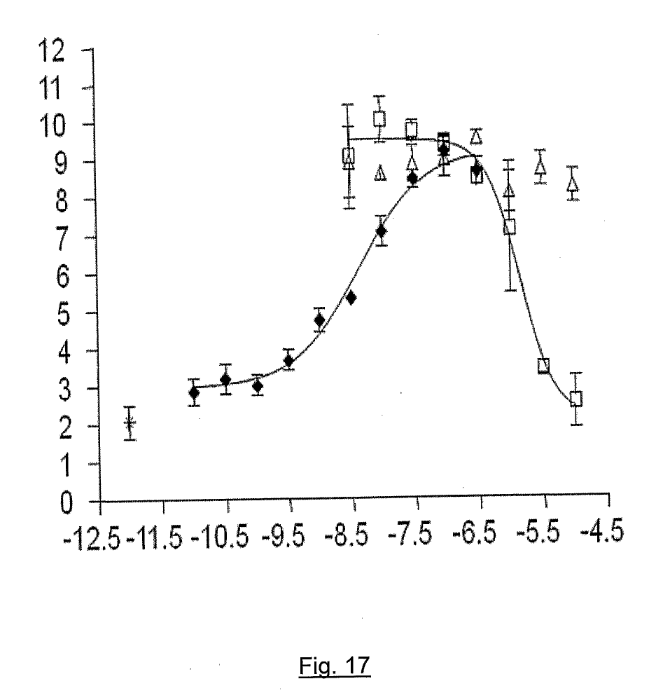

FIG. 17 is a graph showing effect of TL1A (♦), etanercept (Δ) and DcR3 (□) on TRADD-EYFP relocation in HeLa TRADD DR3 recombinant cells. The background (milieu) is shown as “*”. The x-axis shows Log [compound] in g/ml, while the y-axis shows spot count per cell;

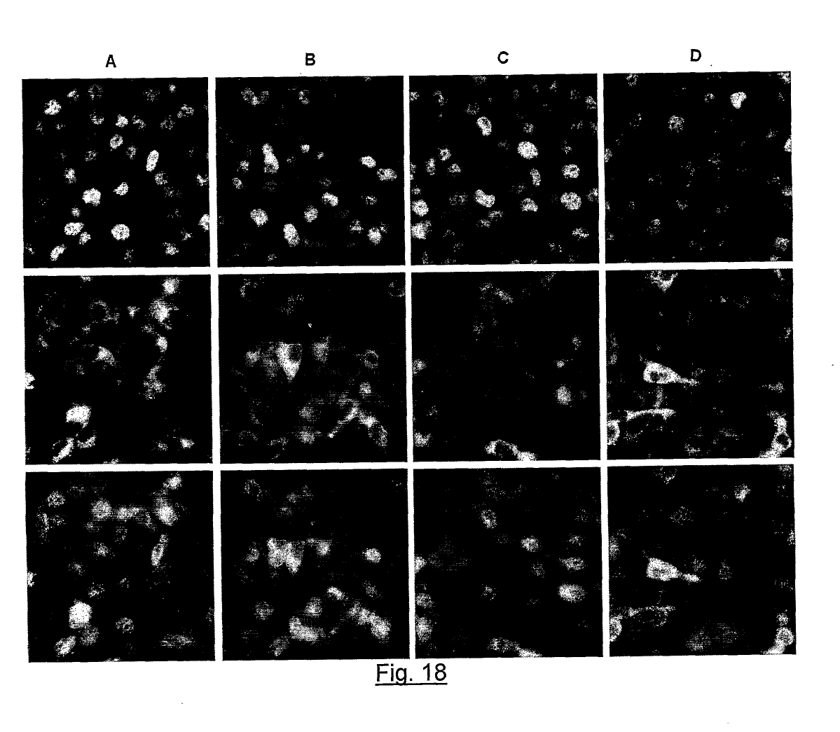

FIG. 18 is a collection of micrographs corresponding to the data depicted in FIG. 17 and showing Hoechst fluorescent nuclear staining (top panels), TRADD-EYFP fluorescence (middle panels) and an overlay of the two (bottom panels) of HeLa TRADD DR3 cells in the presence of medium (column A), 1 μg/ml TL1A (column B), 300 ng/ml TL1A and 10 μg/ml DcR3 (column C), and 300 ng/ml TL1A and 10 μg/ml etanercept (column D);

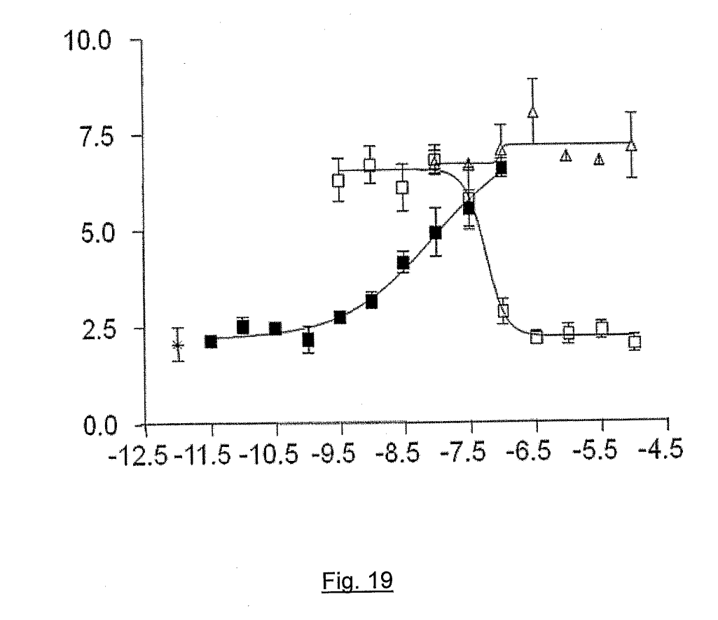

FIG. 19 is a graph showing effect of TNF (♦), etanercept (□) and DcR3 (Δ) on TRADD-EYFP relocation in HeLa TRADD DR3 recombinant cells. The background (milieu) is shown as “*”. The x-axis shows Log [compound] in g/ml, while the y-axis shows spot count per cell;

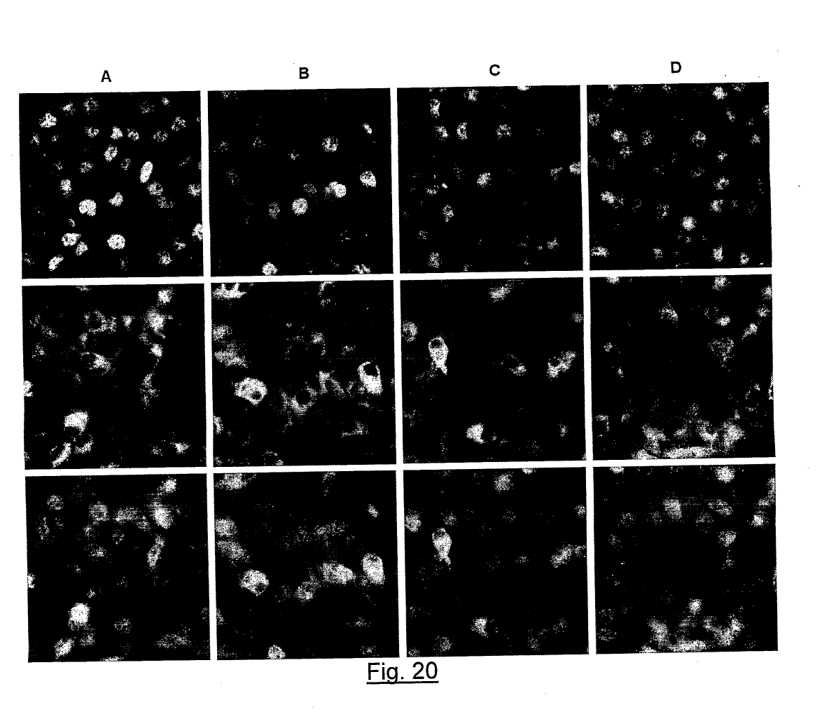

FIG. 20 is a collection of micrographs corresponding to the data depicted in FIG. 19 and showing Hoechst fluorescent nuclear staining (top panels), TRADD-EYFP fluorescence (middle panels) and an overlay of the two (bottom panels) of HeLa TRADD DR3 cells in the presence of medium (column A), 100 ng/ml TNF (column B), 100 ng/ml TNF and 10 μg/ml etanercept (column C), and 100 ng/ml TNF and 10 μg/ml DcR3 (column D).

An example of a method (or “accessory protein relocation assay” [APRA]) according to the invention is illustrated schematically in FIG. 1. Here, an endogenous receptor such as TNFR1 (1) located in the plasma membrane (2) is activated by TNF (3) to form an activated TNFR1 (4). Activated TNFR1 then recruits a modified and detectable accessory protein according the invention such as TRADD-EYFP (5; SEQ ID NO: 18), in the presence of other accessory proteins (such as RIP1; not shown) to form a receptor complex (6). The cellular location and numbers of this clustered receptor complex is visualisable and quantifiable, for example using optical microscopy. The effect of an agent which modulates a signalling pathway mediated by TNF can be determined by comparing the quantity and/or cellular location of the receptor complex in the presence and absence of the agent and optionally in the presence and absence of TNF.

### EXAMPLE 1

In embodiments of the invention set out in this example, gene constructs comprising a combination of TRADD (SEQ ID NO: 1) fused to EYFP (SEQ ID NO: 13) in either order were made. The nucleotide sequence of the fused genes is shown in SEQ ID NO: 15 and SEQ ID NO: 17 below, which encode the fusion proteins of SEQ ID NO: 16 and SEQ ID NO: 18, respectively. These nucleotide sequences were subcloned using the procedure outlined in FIGS. 2 and 3 to create expression vectors pTREhyg-EYFP-TRADD and pTREhyg-TRADD-EYFP which allow expression of the fusion proteins in mammalian cells.

In further detail, the procedure outlined in FIGS. 2 and 3 was as follows. TRADD (SEQ ID NO: 1) was amplified by PCR using HEK293 cells cDNA as template and specific primers 5′-TRADD-SpeI (SEQ ID NO: 3) and 3′-TRADD-NheI (SEQ ID NO: 4), and subcloned into pCR™II-TOPO® (Invitrogen Life Sciences). EYFP (SEQ ID NO: 13, encoding an EYFP polypeptide of SEQ ID NO: 14) was amplified by PCR using pEYFP (Clontech Laboratories) as template and specific primers 5′-EYFP-HindIII (SEQ ID NO: 5) and 3′-EYFP-SpeI (SEQ ID NO: 6), or 5′-EYFP-NheI (SEQ ID NO: 9) and 3′-EYFP-EcoRV (SEQ ID NO: 10), and subcloned into pCR®II-TOPO® already containing TRADD (SEQ ID NO: 1). Then, fusion gene EYFP-TRADD was amplified using specific primers 5′-EYFP-kozak-MluI (SEQ ID NO: 7) and 3′-TRADD-stop-SalI (SEQ ID NO: 8) and subcloned into pTRE2hyg Vector (Clontech Laboratories) as shown in FIG. 2. Similarly the fusion gene TRADD-EYFP was amplified using specific primers 5′-TRADD-kozak-MluI (SEQ ID NO: 11) and 3′-EYFP-stop-SalI (SEQ ID NO: 12) and subcloned into pTRE2hyg Vector (Clontech Laboratories) as shown in FIG. 3. PCR amplification of genes was typically made with 30 cycles consisting of a denaturation step at 95° C. for 1 min, annealing of primers to template at 55° C. for 1 min, and an extension step at 72° C. for 2 min using Taq DNA polymerase. The 30 cycles of the PCR reaction were preceded by a 5 min denaturation step, and ended with a 10 min extension step.

Restriction enzymes (HindIII, SpeI, MluI, SalI, NheI, EcoRV) were obtained from Promega Corporation. Chemically competent E. coli bacteria employed for amplification of plasmids were One Shot® TOP10 (Invitrogen Life Sciences) and JM-109 (Promega Corporation).

The pTREhyg-EYFP-TRADD and pTREhyg-TRADD-EYFP expression vectors were stably transfected using PolyFect® Transfection Reagent (Qiagen), according to the manufacturer's protocol, into HeLa Tet-On Cell Line cells (Clontech Laboratories, Inc.). This cell line is a neomycin resistant human cervical epithelioid carcinoma cell line transformed with a modified version of pTet-On which has a nuclear localization signal fused to the N-terminus (pUHD172-1neo).

Intracellular location of EYFP-TRADD and TRADD-EYFP was determined using fluorescence microscopy. Using a fluorescence microscope, cells are illuminated with light of a specific wavelength which is absorbed by the EYFP, causing it to emit longer wavelengths of light (of a different colour than the absorbed light). The illumination light is separated from the much weaker emitted fluorescence through the use of an emission filter, and an image of the cell created.

In this example, it was observed that in untreated HeLa cells stably transfected with the expression vectors pTREhyg-EYFP-TRADD or pTREhyg-TRADD-EYFP, fluorescently tagged TRADD (i.e. TRADD fused to EYFP) was found diffusely in the cytoplasm of cells (FIG. 4, control). However upon addition of TNF (i.e. recombinant human TNF-α from Peprotech, Cat N° 300-01A) to cells, the fluorescently tagged TRADD repositioned (or relocalised) into punctuate structures in the cytoplasm or at the plasmic membranes of cells (FIG. 4, with TNF).

Using a small molecule antagonist of TNF characterised previously (R-7050; FIG. 5), we then showed that relocalisation of the fluorescently tagged TRADD from cytoplasm to punctuate structures was blocked in the presence of the antagonist (FIG. 6). This showed that the cellular repositioning of TRADD (as a fusion protein with EYFP) was TNF-dependent.

We then used an automated microscope system (a Thermo Scientific Cellomics ArrayScan HCS Reader) as an automated fluorescence microscopic imaging system designed for high content screening and high content analysis. The instrument features include optics by Carl Zeiss, broad white-light source, 12-bit cooled CCD camera, and controller software. The instrument is designed to work with image analysis modules (Thermo Scientific BioApplications) that automatically convert images into numeric data that capture changes in cell size, shape, intensity, and other properties. The system allows: high content screening, analysis of cells containing fluorescent reporter molecules in an array of locations, treating the cells in the array of locations with one or more test compounds, imaging numerous cells in each location with fluorescence optics, and converting the optical information into digital data. This digital data was then used to determine the positioning, distribution, environment or activity of the fluorescently labelled reporter molecules (EYFP-TRADD or TRADD-EYFP) in the transfected HeLa cells and the distribution of the cells. This information was analysed in terms of a positive, negative or null effect of each test compound being examined. This system allowed differences between untreated and treated cells to be measured, for example by counting the number of fluorescent spots (rather than diffuse fluorescence) in cells.

When adding increasing doses of TNF, a dose-dependent relocalisation of the fluorescently tagged TRADD was observed. Using GraphPad Prism (v. 4.01, San Diego, Calif., USA), a dose-response curve was generated (FIG. 7). Percentage responders (y-axis values in FIGS. 7 and 8) were determined as the number of cells with more than five spots, which is an alternative but qualitatively similar measurement to spot count per cell (see below). From FIG. 7, the calculated EC₅₀ TNF was 32.7 ng/ml. Similarly, inhibition of the TNF effect in presence of a reference antagonist compound such as R-7050 (FIG. 5) was quantifiable (see FIG. 8). From FIG. 8, the calculated EC₅₀ TNF was 15.3 ng/ml and the IC₅₀ R-7050 on 1000 ng/ml TNF was 2.1 μM.

The large molecule inhibitor etanercept (Enbrel®, from Amgen, Inc.) was also found to block the TNF-induced relocalisation of TRADD-EYFP (FIG. 9), confirming that the method of the invention can be used to detect or identify small and large inhibitors of TNF signalling. In FIG. 9, the calculated EC₅₀ TNF was 10.5 ng/ml and the IC₅₀ etanercept (TNF dose=300 ng/ml) was 2.74 μM.

As shown in FIG. 10, fluorescence spots per cell were quantified in a 384-well setting on cells treated with TNF, in the presence or absence of etanercept (with cell medium—a background count—shown in plot C). This allowed calculation of the “Z′-factor” of the assay, which is a statistical measure used to evaluate a high-throughput screening (HTS) assay. A score close to 1 indicates an assay is ideal for HTS and a score less than 0 indicates an assay to be of little use for HTS (see Zhang et al., 1999, J. Biomol. Screen. 4: 67-73). Four parameters needed to calculate the Z′-factor are: mean (μ) and standard deviation (a) of both positive (p) and negative (n) control data (μₚ, σₚ, μₙ, σₙ, respectively). Using the formula:

Z factor=1−[3×(σₚ+σₙ)/|μₚ−μₙ|],

the Z′-factor for the assay results shown in FIG. 10 was calculated to be 0.42. The Z′-factor calculation demonstrated that although this assay could be optimised further, the method of the invention is validated for use in HTS.

### EXAMPLE 2

In another embodiment of the invention, the fluorescently labelled reporter molecule EYFP-TRADD was transformed into HeLa cells as described in Example 1 to produce clone #49-E2 cells. The following experimental procedure to detect the reporter molecule in the presence of absence of a “test” compound (or molecule, as described below) was conducted.

On day 1, 10³ cells per well were plated in thin glass bottom 384 wells plate in 50 μl of culture media (DMEM glutamax, 10% FBS, peni-strep, geneticin, hygromycin). The cells were incubated overnight at 37° C. On day 2, media was aspirated, and 35 μl of warm DMEM, 2, 5% FBS, HEPES 10 mM added. Then, 15 μl of 4× the test compound diluted in DMEM, 2, 5% FBS, HEPES 10 mM was added to cells using a Biomek laboratory automation workstation (Beckman-Coulter). This was incubated at room temperature for 1.5 min. Then, 20 μl of 4×TNF (or TL1A or IL1b) diluted in DMEM, 2, 5% FBS, HEPES 10 mM was added to the cells using the Biomek workstation, and the cells were incubated at 37° C. for 15 min. The cells were washed three times with PBS 1× using an Embla 384 Cell Washer (Molecular Devices). Cells were fixed by adding PBS 1×, 5% PFA+Hoescht 0.2 μg/ml using a Multidrop 384 dispensor (Thermo Scientific; 30 μl/well) followed by an incubation for 20 min at room temperature in the dark. After a further three washes of the cells with PBS 1× using the Embla 384 Cell Washer, 60 μl per well of PBS 1×, 1% peni-strep was added. The plate was covered with a transparent sealing film. Readings were then taken on the Scientific Cellomics ArrayScan HCS Reader automated microscope system as used in Example 1.

The general performance characteristics of the method can be seen in FIG. 11, where as shown in Example 1a TNF dose-dependent relocalisation of the EYFP-TRADD was observed. The window signal/noise level was typically greater than 15×. Z′-factor (EC₈₀) for the results shown in FIG. 11 was calculated to be greater than 0.6.

Selectivity of the method was shown by comparing the effect of TNF-dependent relocalisation of the fluorescently tagged TRADD with other compounds or molecules, viz. TL1A (Recombinant Human TL1A/TNFSF15 from R&D Systems, Cat N° 1319-TL), IL1β (Recombinant Human IL1β from Peprotech, Cat N° 200-01B), CD40L (Fc [human]: CD40L [human] from Apotech Corporation, Cat N° APO-50N-057-C010), TRAIL (recombinant human TRAIL from Millipore, Cat N° GF092), anti-FAS (human, activating anti-FAS, clone CH11 from Millipore, Cat N° 05-201), Poly I:C (Polyinosinic-polycytidylic acid potassium salt from SIGMA, Cat N° P9582, CAS Number 31852-29-6) and LPS (Lipopolysaccharides from Escherichia coli 055:B5, cell culture tested, γ-irradiated from SIGMA, Cat N° L6529). From the results shown in FIG. 12, it can be seen that the assay is specific to TNF-induction, as relocalisation of the reporter molecule EYFP-TRADD (indicating formation of TNFR1-containing complex I) is not effected by the other compounds or molecules.

FIG. 13 confirms the data shown in FIG. 10 of Example 1, that the large molecule inhibitor etanercept blocks the TNF-induced relocalisation of EYFP-TRADD.

The effect of blocking downstream signalling following TNF induction of TNFR1 was investigated using various inhibitors (in the presence of TNF). FIG. 14 shows that in the presence of increasing concentrations of Z-IETD-FMK, an irreversible caspase 8 inhibitor (obtained from Tocris, Cat N° 2170), there was no dissociation of clusters detectable by EYFP-TRADD. As noted in the prior art discussion above, caspase 8 is involved in the formation of complex IIA and IIB that form under certain circumstances when complex I comprising TNF, TNFR1, TRADD, RIP1, cIAPS 1 and 2, and TRAF2 or TRAF5 dissociates. Inhibition of caspase 8 thus did not impact on the levels of TRADD-containing clusters detectable by EYFP-TRADD. Similarly, when increasing levels of the IKK inhibitor IKK16 (obtained from Tocris, Cat N° 2539) was added to the HeLa cells transformed with EYFP-TRADD in the presence of TNF, no change in detectable EYFP-TRADD clusters were observed (FIG. 15). Finally, in the presence of Zygosporium masonii cytochalasin D (obtained from SIGMA, Cat N° C2618, CAS Number 22144-77-0), a reduction in detectable EYFP-TRADD clusters were observed as the concentration of cytochalasin D increased, but this reduction ceased at the higher concentrations of cytochalasin D (FIG. 16). Cytochalasin D is an actin polymerisation inhibitor which has also been shown to affect TNF production, for example LPS-induced TNF production.

These data using downstream signalling inhibitors show that the methods of the present invention are detecting proximal adaptor proteins primarily recruited upon receptor activation—in this embodiment, TRADD recruitment upon TNF activation of TFNR1—and that downstream signalling inhibition does not impact on the initial stage of receptor activation. The methods are thus useful in monitoring early stages of TNF-induced activation of relevant receptors (such as TFNR1).

### EXAMPLE 3

In a further example of the present invention, a method involving the TL1A-activated DR3 receptor was developed. The TRADD-EYFP HeLa clones comprising the TRADD-EYFP reporter molecule transfected into HeLa cells as described in Example 1 was employed. The DR3 receptor (cDNA sequence shown in SEQ ID NO: 19 and encoded protein shown in SEQ ID NO: 20) which had been cloned in the vector pCR3.1 (Invitrogen) was transferred into the vector pTre2 (Clontech) using the Gateway® cloning technique with the primers Attb1 Forward primer (SEQ ID NO: 21) and Attb2 Reverse primer (SEQ ID NO: 22), yielding a pTRE2-DR3 expression vector (SEQ ID NO: 23). This expression vector was then transfected into the TRADD-EYFP HeLa clones to produce a double transformants designated HeLa TRADD DR3.

Monitoring and quantification of the TRADD-EYFP reporter molecule was conducted as described in Examples 1 and 2 above. When adding increasing doses of TL1A, a dose-dependent relocalisation of the TRADD-EYFP molecule to form distinct spots was observed (see FIG. 17). The calculated EC₅₀ of TL1A was 4.3 ng/ml. As shown in FIG. 17, this TL1A-induced relocalisation was found to be dose-dependently inhibited in the presence of 300 ng/ml TL1A by the DR3 inhibitor DcR3 (Recombinant Human DcR3/TNFRSF6B/Fc Chimera, CF obtained from R&D Systems, Cat N° 142-DC; IC₅₀=1.41 μg/ml) but not by the TNF inhibitor etanercept. The data of FIG. 17 are depicted graphically in FIG. 18.

Based on further experiments shown in FIGS. 19 and 20, we determined that co-expression of TRADD-EYFP and DR3 in the HeLa TRADD DR3 cells did not alter the TNF effect previously observed in Examples 1 and 2. In other words, in the HeLa TRADD DR3 cells, TNF still dose-dependently induces TRADD relocalisation to distinct spots (here, EC₅₀=9.7 ng/ml). This TNF-induced relocalisation is dose-dependently inhibited in the presence of 100 ng/ml TNF by the TNF inhibitor etanercept (IC₅₀=53.8 ng/ml) but not by the D3 inhibitor DcR3.

The data in this example confirm that the TRADD-linked reporter molecules of the present invention can be used to specifically monitor different receptors for TNF (including TNF homologues or TNF-like molecules) in which TRADD is involved. Thereby, agents which monitor various signalling pathways mediated by the TNF (including TNF homologues or TNF-like molecules) can be identified.

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

The sequences set out in the Sequence Listing below form part of the present description.

### SEQUENCE LISTING FREE TEXT

Synthetic oligonucleotide (SEQ ID NOs 3-12, 21 and 22);

### Synthetic EYFP DNA (SEQ ID NO: 13);

### Synthetic Construct (SEQ ID NOs 14, 16, 18);

Synthetic EYFP-TRADD construct (SEQ ID NO: 15);
Synthetic TRADD-EYFP construct (SEQ ID NO: 17);
Synthetic pTRE2-DR3 expression vector (SEQ ID NO: 23).

1. A method of identifying an agent that modulates a signalling pathway mediated by tumour necrosis factor (TNF), comprising the steps of: (i) providing a host cell comprising TNF receptor 1 (TNFR1)-associated death domain (TRADD) linked to a reporter molecule; (ii) determining at least one cellular characteristic detectable by the reporter molecule in the host cell in the presence of TNF and in the absence of a candidate modulator; (iii) contacting the host cell with the candidate modulator; (iv) determining the at least one cellular characteristic in the presence of the candidate modulator and TNF; and (v) identifying whether the candidate modulator is an agent that modulates a signalling pathway mediated by TNF, in which a change in the at least one cellular characteristic in the presence of said candidate modulator relative to the at least one cellular characteristic in the absence of the candidate modulator identifies the candidate modulator as an agent that modulates a signalling pathway mediated by TNF.

2. The method according to claim 1, in which the signalling pathway is mediated by TNF binding to TNFR1.

3. The method according to claim 1, further comprising the step of determining the at least one cellular characteristic in the absence of TNF and in the presence or absence of the candidate modulator.

4. The method according to claim 1, in which the host cell comprises a recombinant nucleic acid having a first sequence encoding a TRADD polypeptide and a second sequence encoding a reporter molecule, in which the encoded TRADD polypeptide and the reporter molecule are linked, for example as a fusion protein.

5. The method according to claim 1, in which the host cell comprises a recombinant nucleic acid having or consisting of the nucleic acid sequence of SEQ ID NO: 15 or SEQ ID NO: 17, or a nucleic acid sequence having at least 60% sequence identity with either of SEQ ID NO: 15 or SEQ ID NO: 17.

6. The method according to either of claims 4 or 5, in which the recombinant nucleic acid encodes a fusion protein comprising or consisting of the amino acid sequence of SEQ ID NO: 16 or SEQ ID NO: 18, or a variant thereof having at least 50% sequence identity with the fusion protein.

7. The method according to claim 1, in which the host cell comprises a polypeptide having or consisting of the amino acid sequence of SEQ ID NO: 16 or SEQ ID NO: 18, or a variant thereof having at least 50% sequence identity with the polypeptide.

8. The method according to claim 7, in which the polypeptide or variant is a fusion protein comprising or consisting of TRADD and EYFP (or functional variants thereof).

9. The method according to claim 1, in which the host cell comprises a polypeptide having or consisting of the amino acid sequence of SEQ ID NO: 2, or a variant thereof having at least 50% sequence identity with the polypeptide, in which the polypeptide or its variant is linked to a reporter molecule.

10. The method according to claim 9, in which the polypeptide comprises or consists of TRADD.

11. The method according to claim 1, in which the agent is a negative allosteric modulator of a signalling pathway mediated by TNF.

12. The method according to claim 1, in which the at least one characteristic is determined in step (iv) up to 60 minutes after both the candidate modulator and TNF are present in the host cell, for example between 1 and 60 minutes, between 5 and 30 minutes, or between 10 and 15 minutes after both are present.

13. The method according to claim 1, further comprising the step of isolating and/or purifying a candidate modulator which is identified as an agent that modulates a signalling pathway mediated by TNF.

14. A method of identifying an agent that modulates binding of TRADD to an associated receptor, comprising the steps of: (i) providing a host cell comprising TRADD linked to a reporter molecule; (ii) determining at least one cellular characteristic detectable by the reporter molecule in the host cell using the reporter molecule in the absence of a candidate modulator; (iii) contacting the host cell with the candidate modulator; (iv) determining the at least one cellular characteristic in the presence of the candidate modulator; and (v) identifying whether the candidate modulator is an agent that modulates binding of TRADD to the associated receptor, in which a change in the at least one cellular characteristic in the presence of said candidate modulator relative to the at least one cellular characteristic in the absence of the candidate modulator identifies the candidate modulator as an agent that modulates binding of TRADD to the associated receptor.

15. The method according to claim 14, in which the associated receptor is TNFR1.

16. The method according to claim 14, in which the associated receptor is any one or the group consisting of DR3, DR4, DR5, DR6, TLR3, TLR4, RIG-like helicase, and interferon-γ receptor.

17. The method according to claim 14, conducted in the presence or absence of a signalling molecule that interacts with the associated receptor.

18. The method according to claim 17, in which the at least one characteristic is determined in step (iv) up to 60 minutes after both the candidate modulator and the signalling molecule that interacts with the associated receptor are present in the host cell, for example between 1 and 60 minutes, between 5 and 30 minutes, or between 10 and 15 minutes after both are present.

19. The method according to claim 14, further comprising the step of isolating and/or purifying a candidate modulator which is identified as an agent that modulates binding of TRADD to an associated receptor.

20. The method according to claim 1 or claim 14, in which the at least one characteristic is cellular positioning (for example, intracellular positioning) of TRADD.

21. The method according to claim 1 or claim 14, in which the reporter molecule is a fluorescent reporter molecule, for example a fluorescent protein (FP) such a green fluorescent protein (GFP), yellow fluorescent protein (YFP) or enhanced YFP (EYFP), a luciferase, or a functional fragment of any of these.

22. The method according to claim 1 or claim 14, in which the reporter molecule is detectable by a fluorescence detector.

23. The method according to claim 22, in which the at least one characteristic is determined using fluorescence microscopy.

24. The method according to claim 1 or claim 14, comprising the step of imaging or scanning multiple host cells.

25. The method according to claim 1 or claim 14, for use in high-content screening.

26. A high-throughput screening (HTS) assay comprising a method according to claim 1 or claim 14.

27. A high-content screening (HCS) assay comprising a method according to claim 1 or claim 14.

28. A combination assay comprising the HTS assay of claim 26 and the HCS assay of claim 27.

29. An agent that modulates a signalling pathway mediated by TNF, in which the agent is detected according to the method of claim 1.

30. An agent that modulates binding of TRADD to an associated receptor, in which the agent is detected according to the method of claim 14.

31. (canceled)
